(19) United States
(12) Patent Application Publication
(10) Pub. No.: US 2022/0249884 A1
(43) Pub. Date: Aug. 11, 2022

(54) FACEPIECE FILTER HAVING OFFSET LAYERED FEATURES

(71) Applicant: LCP Medical Technologies, Inc., Rogers, MN (US)

(72) Inventor: James Rathburn, Rogers, MN (US)

(21) Appl. No.: 17/661,216

(22) Filed: Apr. 28, 2022

Related U.S. Application Data
(63) Continuation-in-part of application No. PCT/US21/29764, filed on Apr. 28, 2021.
(60) Provisional application No. 63/016,441, filed on Apr. 28, 2020.
(60) Provisional application No. 63/142,422, filed on Jan. 27, 2021.
(60) Provisional application No. 63/181,287, filed on Apr. 29, 2021.
(60) Provisional application No. 63/182,484, filed on Apr. 30, 2021.

(51) Int. Cl. A62B 23/02; A41D 13/11

(52) U.S. Cl. CPC A62B 23/02; A61M 16/0616; A41D 13/1192

(57) ABSTRACT

A facepiece comprising a main body having a geometry configured to fit on a human face and cover the human's mouth and nose is provided. The main body defines an interior cavity between the main body and the human's face. The facepiece also includes one or more straps extending from the main body and configured to extend around the ears or head of a human such that the main body is held on the human's face. The main body includes a filter defining a plurality of airflow torture paths extending therethrough between the interior cavity and an external environment. The filter includes three or more layers of airflow path defining features, each airflow path defining feature providing a passageway for air to pass therethrough. The path defining features in adjacent layers are fluidly coupled but offset to form the airflow torture paths.

### RELATED APPLICATIONS

This application is a continuation-in-part of PCT patent application no. PCT/US21/29764 filed on Apr. 28, 2021 and titled “ANTIPATHOGEN RESPIRATOR”, which claims the benefit of U.S. Provisional Application No. 63/016,441, filed on Apr. 28, 2020, entitled “Antipathogen Protective Facial Mask with Active Electronics Options”, and U.S. Provisional Application No. 63/142,422, filed on Jan. 27, 2021, entitled “PLANAR ANTI-PATHOGEN STRUCTURE SUSPENDED WITHIN THE AIR FLOW PATH WITHIN A PROTECTIVE FACIAL MASK OR RESPIRATOR”. This application also claims the benefit of U.S. Provisional Application No. 63/181,287, filed on Apr. 29, 2021, entitled “MD1004 ANTIPATHOGEN PROTECTIVE FACIAL MASK WITH ACTIVE ELECTRONICS OPTIONS” and U.S. Provisional Application No. 63/182,484, filed on Apr. 30, 2021, entitled “7000-1 PROTECTIVE FACIAL MASK WITH AIRFLOW DIRECTED FILTRATION AND ANTI-PATHOGEN FEATURE OPTIONS”. Each of the foregoing applications is hereby incorporated herein by reference.

### BACKGROUND

Traditional facial masks used for medical situations range from basic cloth patches that cover the mouth and nose, to more elaborate formed structures that have fibrous structures that create a filter effect for air that is breathed in by the user as well as exhaled by the user. In some cases, the filter effect is directed at protecting the user from inhaled pathogens or contaminants, and in some cases the intent is to prevent the user from exhaling pathogens or infections particles. In general, most commercially available respirators are intended for a one time use and discarded. They are difficult or expensive to sterilize and return to as new condition. In addition, most if not all commercially available respirators are designed and used to reduce exposure to pathogens and do not attack the potential pathogens themselves.

### BRIEF DESCRIPTION

Embodiments described herein provide for a facepiece comprising a main body having a geometry configured to fit on a human face and cover the human's mouth and nose. The main body defines an interior cavity between the main body and the human's face. The facepiece also includes one or more straps extending from the main body and configured to extend around the ears or head of a human such that the main body is held on the human's face. The main body includes a filter defining a plurality of airflow torture paths extending therethrough between the interior cavity and an external environment. The filter includes three or more layers of airflow path defining features, each airflow path defining feature providing a passageway for air to pass therethrough. The path defining features in adjacent layers are fluidly coupled but offset to form the airflow torture paths.

### DRAWINGS

Understanding that the drawings depict only exemplary embodiments and are not therefore to be considered limiting in scope, the exemplary embodiments will be described with additional specificity and detail through the use of the accompanying drawings, in which:

FIGS. 1A and 1B are perspective views of an example facepiece that includes layers of airflow path defining features offset from one another to form airflow torture paths therethrough;

FIG. 2 is a front view of an example sheet of polymer on which a plurality of airflow path defining features are formed;

FIGS. 3A, 3B, and 3C are enlarged views of example airflow path defining features that can be formed in the sheet of FIG. 2;

FIGS. 3D, 3E, and 3F are cross-sectional views of a sheet having the features of FIGS. 3A, 3B, and 3C therein;

FIGS. 4A and 4B are front and cross-sectional views of an example sheet of polymer defining higher aspect ratio apertures;

FIGS. 5A, 5B, and 6A, 6B are cross-sectional and front views of an example sheet during steps in a process to create small apertures;

FIGS. 7A, 7B, and 8A, 8B are cross-sectional and front views of example steps in a process of creating airflow torture paths with the multiple sheets of airflow path defining features;

FIG. 8C is an exploded view of an example stack-up of the sheets of FIGS. 7A-8B;

FIGS. 9A and 9B are a front view and a cross-sectional view of example steps in forming a main body in which a filter is a monolithic structure with the remainder of the main body;

FIGS. 10A and 10B are front and cross-sectional views of another example sheet that can be used in a filter as described herein;

FIGS. 11A and 11B are front and cross-sectional views of yet another example sheet that can be used in a filter as described herein;

FIGS. 12A and 12B are front and cross-sectional views of another example stack-up of sheets defining airflow torture paths therethrough;

FIG. 12C is a cut-away view of the stack-up of FIG. 12B;

FIGS. 13A and 13B are front and cross-sectional views of yet another stack-up;

FIGS. 14A and 14B are front and cross-sectional view of yet another stack-up;

FIGS. 15A and 15B are front and partial cross-sectional views of still another stack-up;

FIGS. 15C, 15D, and 15E are front views of example sheets in the stack-up of FIGS. 15A and 15B;

FIG. 16A is front view of an example facepiece having a filter formed in polymer sheets that are monolithic with the main body;

FIG. 16B is a cross-sectional view of a facepiece having an exchangeable filter cartridge removably secured thereto;

FIGS. 17A and 17B are a front view and a cross-sectional view of an example main body having embedded ultraviolet LEDs and other integrated circuit devices therein;

FIG. 18 is an exploded view of air chambers define an example stack-up;

FIGS. 19A-C are a front view and two cross-sectional view of another example respirator;

FIGS. 20A and 20B are front views of an example respirator having a polymer strap attached thereto;

FIGS. 21A-C are a front view, an enlarged view, and a cross-sectional view of an example respirator having relief cuts defined therein;

FIGS. 22A and 22B are a front view and a cross-sectional view of an example off-the-shelf respirator having a cartridge filter;

FIGS. 23A-C are front views and a cross-sectional view of an example respirator having a strap with embedded circuits therein;

FIGS. 24A and 24B are cross-sectional views of yet another example respirator;

FIGS. 25A-C are front views and cross-sectional views of another example respirator;

FIGS. 26A-D are perspective views of an example base frame of a main body to which a cartridge can be removably secured;

FIGS. 27A and 27B are exploded views of an example cartridge for use in any of the cartridge respirators described herein;

FIGS. 28A-C are perspective views of an example compliant face rim that can be attached to the base frame;

FIGS. 29A and 29B are a front view and a cross-sectional view of another example filter cartridge;

FIGS. 30A and 30B are cross-sectional views of an example of a cloth stack that can be used as filter material on a filter cartridge;

FIG. 31 is a cross-sectional view of a filtering portion that can be used in a filter cartridge;

FIGS. 32A-C are front views of example components for the filtering portion of FIG. 31;

FIG. 33 is a cross-sectional view of another example filtering portion;

FIGS. 34A and 34B is a front view of the pinwheel spacer layer;

FIG. 34C is a side cross-sectional view of the filter portion of FIG. 33;

FIGS. 35A-C are a front view, an enlarged view, and a cross-sectional view of another example copper flex layer;

FIG. 36 is another example of a base frame of a respirator;

FIGS. 37A-D are perspective views of example detachable pivoting clips for detachably connecting head straps to the base frame;

FIGS. 38A-D are illustrations of an example filter cartridge;

FIG. 39 is an exploded view of another example filter cartridge;

FIGS. 40 and 41 are cross-sectional views of two example filters that are adhered to one or more apertures in a facepiece;

FIG. 42 is a cross-sectional view of another tapeable filter;

FIG. 43 is a cross-sectional view of yet another tapeable filter;

FIGS. 44 and 45 are a front view of other example face shields having a tapeable filter;

FIGS. 46A-F are other example respirators having slide-in filter cartridges;

FIG. 47 is a side view of another example mask;

FIG. 48 is a front view of an example stiffener for the mask of FIG. 47;

FIG. 49 is an example of antipathogen panel that can be mounted to the stiffener of FIG. 48;

FIGS. 50A and 50B are a front view and a cross-sectional view of a portion of an example stiffener;

FIG. 51-53 are front views of other example stiffeners;

FIG. 54 is a front view of another example antipathogen panel;

FIG. 55 is a front view of an example spacer frame that can be disposed on the antipathogen panel of FIG. 54;

FIGS. 56A and 56B are cross-sectional views of portions of other example stiffeners;

FIG. 57 is a front view of an example inner carrier for the stiffeners of FIGS. 56A and 56B;

FIG. 58 is a front view of an example foam adhesive for the stiffeners of FIGS. 56A and 56B;

FIG. 59 is a front view of an example outer carrier for the stiffeners of FIGS. 56A and 56B; and

FIG. 60 is a side view of an example mask that includes a clear panel.

### DETAILED DESCRIPTION

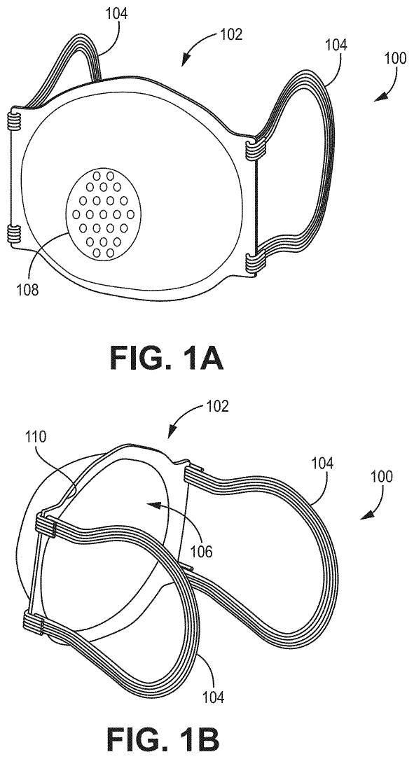

FIGS. 1A and 1B are perspective views of an example facepiece 100 having a filter 108b that defines a plurality of airflow torture paths therethrough. The example facepiece 100 is a standalone respirator designed to be worn by a human user and to filter air flowing to and/or from the user's mouth and nose. In other examples, the facepiece 100 can be a standalone facial mask (e.g., a cloth mask) or a mask configured to be coupled to a supply of gas, such as a mask for a continuous positive airway pressure (CPAP) machine or ventilator.

Facepiece 100 includes a main body 102 having or more straps 104 extending therefrom. The main body 102 defines an interior cavity 106 and is configured to cover a mouth and a nose of a user. In this example, the main body 102 is sufficiently rigid to maintain its overall shape during normal care on and off of a user's face. Example materials that are sufficiently rigid include melt blown GSM materials used in conventional facial masks and respirators, polymers, including thermoelastic polymers such as liquid crystal polymer (LCP), polyimide, polyolefin, Polycarbonate, PEI, Acrylic, Silicone, Neoprene, and others. Liquid Crystal Polymer (LCP) is a thermopolymer material that can be shaped, formed, and molded. LCP is impervious to moisture and is biocompatible. In still other examples, an insufficiently rigid material (e.g., cloth fabric) can be used along with a rigid frame to provide an overall rigid geometry for the body 102. The interior cavity 106 is sized such that the user's nose and mouth fit within the cavity 106. In this example, the main body 102 has a generally concave geometry defining a single depression large enough to cover both the user's nose and mouth. In other examples, other geometries can be used.

In one example, main body 102 has a construction based upon Polycarbonate or Acrylic polymers that provide mechanical infrastructure to incorporate multiple components and features while being optically clear to allow for the users face and facial expressions to be viewed while conventional respirators block the view of the face. These polymers will typically yield a rigid structure but depending on the design the facepiece 100 could be made flexible or partially flexible for reasons such as flat storage or distribution.

The general shape and appearance of the main body 102 and overall respirator can be generally curvilinear and in basic terms serves as the structure that holds and presents the filtration and anti-pathogen structure at the proper location within the airflow path. The structure also serves as the skeleton for arranging and attaching various components that provide features such as facial sealing, respirator retention on the user's head, filter replacement, electronics integration, filter attachment etc.

In still other examples, the main body 102 can be flexible in nature, such that main body 102 has no rigid three-dimensional shape. In such examples, the main body 102 takes a generally concave geometry that covers the user's nose and mouth upon being strapped to the user's face. An example of such a flexible material is a cloth fabric, for example, composed of cotton, nylon, wool, silk, or a combination thereof.

The one or more straps 104 are configured to hold the facepiece 100 onto the face of the user. In this example, the one or more straps 104 are configured to wrap around the ears of the user, but other straps can be used such as one or more straps extending around the back and/or top of the user's head. In use, the facepiece 100 is configured to be placed over the mouth and nose of a user such that the outer rim 110 of the main body 102 contacts the user's face around the mouth and nose.

The facepiece 100 provides one or more airflow paths for air to flow between the interior cavity 106 and the external environment while the facepiece 100 is being worn. Accordingly, the airflow paths provide a path for a user's breath to enter and/or exit the interior cavity 106 as the user inhales and/or exhales while wearing the facepiece 100. In some examples, the main body 102 is composed of materials that provide airflow paths across the entirety of the main body 102, such as is the case with cloth fabric or melt blown GSM material. In other examples, the main body 102 is composed of materials, such as a thermoelastic polymer, that blocks airflow between the interior cavity 106 and the external environment. In such other examples, the main body 102 defines one or more passages through the impervious material, wherein the passages which provide airflow paths from the interior cavity 106 to the external environment.

As mentioned above, the main body 102 of the facepiece 100 includes a filter 108 that defines a plurality of airflow torture paths for capturing particles traversing therethrough. In an example, the filter 108 and the main body 102 can be a monolithic structure, such that the main body 102 and filter 108 are formed of the same material in the same process. In other examples, the filter 108 can be in a cartridge that is removably connected to the remainder of the main body 102. Such a cartridge can be removed and replaced with another cartridge to provide a fresh filter in the facepiece 100. More detail regarding the monolithic structure embodiment and the cartridge embodiment are provided below.

In either embodiment, the filter 108 can be composed of a generally rigid polymer that maintains its basic geometry during use on a human's face. Sufficiently rigid polymer can flex minimally in response to hand force thereon but return to its previous geometry after the force is released. The airflow torture paths can be formed through the polymer by including multiple layers of airflow path defining features that allow air flow through the otherwise solid polymer. The airflow path defining features can have different forms including apertures and flexible flaps that allow air to flow through the polymer. Multiple layers of the airflow path defining features can be stacked on top of one another and can be offset with one another to form the weaving torture path through the polymer. The features are disposed to define torture paths with sufficiently small openings and direction changes for air passing therethrough to capture and filter out particles in the air flowing therethrough.

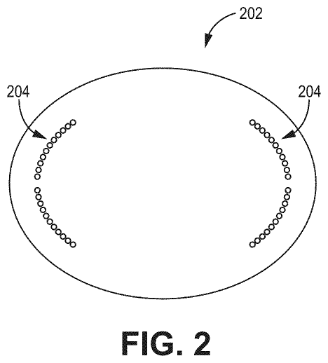

FIG. 2 is a front view of an example sheet 202 of polymer on which a plurality of airflow path defining features 204 are formed. In an example, the sheet 202 is a thermopolymer, such as LCP. The airflow path defining features 204 can provide passageways for air to traverse from one side of the sheet 202 to the other side. Although a certain number and position of airflow path defining features 204 are shown, any number or pattern of features 204 can be used. The example features 204 shown in FIG. 2 have been reduced in number and increased in size for clarity. In an example, the sheet 202 is less than 100 microns thick.

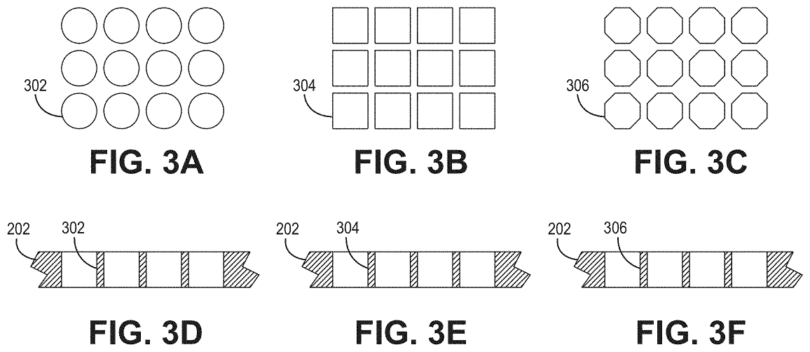

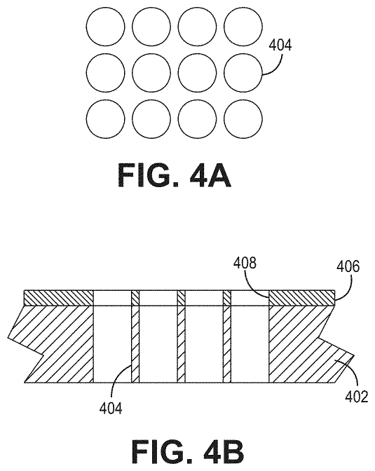

FIGS. 3A, 3B, and 3C are enlarged views of example airflow path defining features 302, 304, 306 that can be formed in the sheet 202. In these examples, the features 302, 304, 306 are apertures that extend through the sheet 202, thereby providing a passageway for air to flow through the sheet 202. FIGS. 3D, 3E, and 3F are cross-sectional views of the sheet 202 showing the apertures 302, 304, 306 extending through the sheet 202. Referring back to FIGS. 3A, 3B, and 3C, the apertures 302, 304, 306 can have different cross-sectional shapes including circular, square, hexagonal and others. The apertures 302, 304, 306 can be formed in any suitable manner including via laser ablation, punching, embossing, or plasma etching. Each of these processes has an aspect ratio limitation where the cross-sectional size of the aperture 302, 304, 306 is relative to the thickness of the sheet 202. With normal processing, a 1:1 aspect ratio is the general rule, such that the aperture in a 25 micron thick sheet 202 drives a 25 micron wide aperture 302, 304, 306. For use in a facial mask application, a 25 micron wide/across (e.g., in diameter) aperture is too large for effective filtering of particles and pathogens. Typical N95 masks are rated for 95% filtering of particles of 1 micron or smaller in size. To create sufficiently small torture paths, at least some of the apertures 302, 304, 306 should be smaller than 25 microns wide/across (e.g., in diameter).

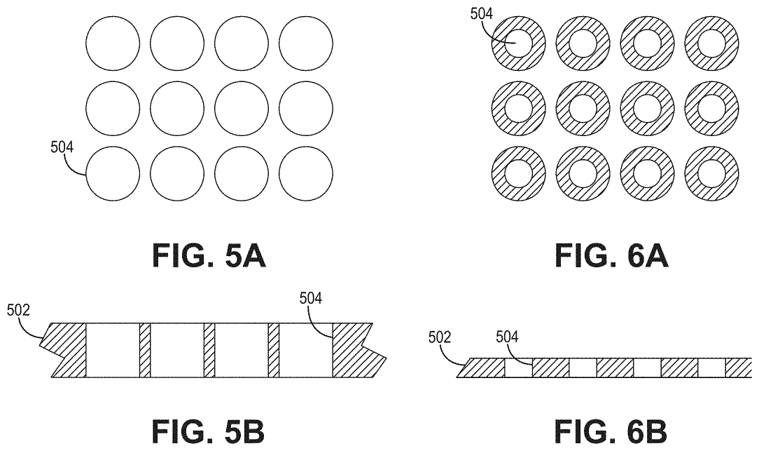

FIGS. 4A and 4B are front and cross-sectional views of an example sheet 402 in which higher aspect ratio apertures 404 can be formed. The apertures 404 having a width smaller than the thickness of the sheet 402 of polymer can be formed by placing (e.g., depositing) a thin copper mask 406 on top of the sheet 402 of polymer and then etching an aperture 408 in the copper mask 406. In an example, the copper mask 406 is less than 10 microns thick and the aperture 408 in the copper mask 406 is less than 25 microns wide. A laser or plasma process can then be applied to the sheet 402 of polymer through the apertures 408 of the copper mask 406 to remove the polymer material of the sheet 402 and create the apertures 404. The laser or plasma process is configured such that the copper mask 406 is not removed and acts to limit the area of polymer that is exposed to the laser or plasma process. The resulting apertures 404 can have a higher aspect ratio enabling the apertures 404 to be smaller in width than the thickness of the sheet 202. After the apertures 404 have been formed the copper mask 406 can be removed or, alternatively, can be left on the sheet 402 during further construction of a filter.

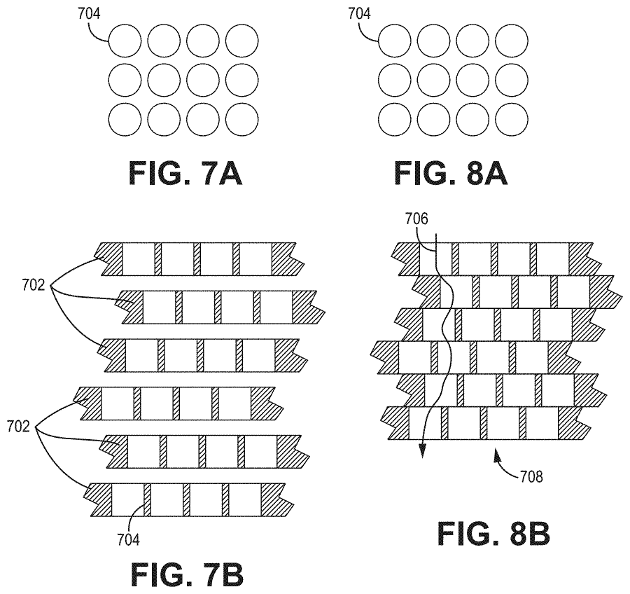

FIGS. 5A, 5B, and 6A, 6B are cross-sectional and front views of an example sheet 502 during steps in another process to create smaller apertures. This process includes creating apertures 504 in a sheet 502 of polymer using the standard processes that create apertures having an aspect ratio in the 1:1 range. Heat and pressure are the applied to the sheet 502 shown in FIGS. 5A and 5B reducing the thickness of the sheet 502 to that shown in FIG. 6B. This reduction of thickness causes the polymer to move inward within the apertures 504 such that the apertures 504 shrink in width as shown in FIGS. 6A and 6B.

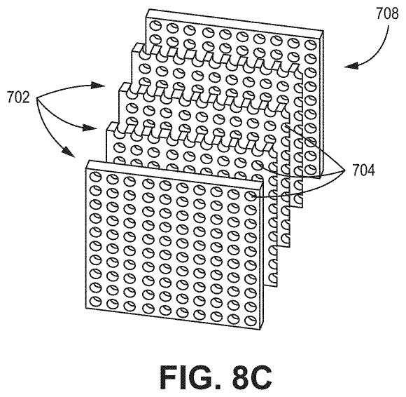

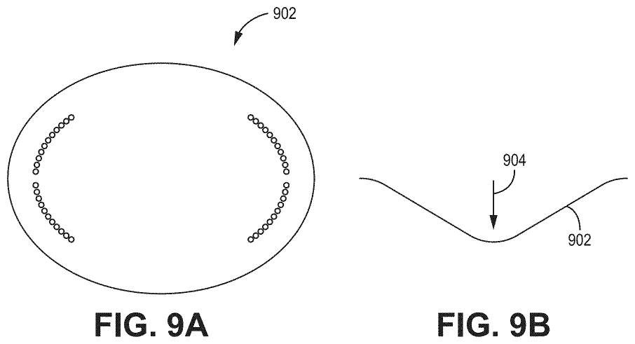

FIGS. 7A, 7B, and 8A, 8B are cross-sectional and front views of example steps in a process of creating airflow torture paths with the multiple sheets 702 of airflow path defining features 704. In this example, the sheets 702 can be sheets having airflow path defining features 704 which are apertures created using any of the above processes described in FIGS. 3A-6B. FIG. 8C is an exploded view of an example stack-up of sheets 702 of airflow path defining features 704. In an example, all sheets 702 can have the same apertures (i.e., geometry and size). In other examples, different size and/or geometry apertures can be used within a single sheet 702 and/or in different sheets 702.

As shown in FIG. 7B, multiple such sheets 702 can be disposed in a stack. The sheets 702 can be disposed in the stack such that adjacent sheets 702 are offset with one another to create airflow torture paths 706 through the resulting stack-up 708. The sheets 702 are disposed such that apertures 704 in one sheet 702 are fluidly coupled but offset with apertures in adjacent sheet(s) 702. This fluid coupling with misalignment creates airflow paths that weave through the stack-up and include partial blockages due to polymer material between apertures 704 of a first sheet 702 partially covering apertures 704 of adjacent sheet(s) 702. As shown in FIG. 8B, the stack of sheets 702 can then be fusion bonded together to create the airflow torture paths 706 through a stack-up 708 of multiple sheets. In an example, the torture paths can be configured to achieve N95 type requirements for particle restriction. In an example, the sheets 702 are composed of a thermopolymer material such as LCP that bonds to itself under heat and pressure. In such an example, the multiple sheets 702 are bonded directly to one other to create the stack-up 708. The sheets 702 can be bonded together across all touching surfaces or, alternatively, can be bonded together only in areas outside of (i.e., that do not include) apertures 704. In an alternative example, a bond layer, can be disposed between adjacent sheets 702 to aid in bonding the sheets 702 together. The bond layer can define one or more large apertures that span the area(s) in which the apertures 704 are disposed so that the bond layer does not block the airflow paths 706 in the stack-up. In such an example, the sheets 702 can be bonded to one another in the area(s) that include the bond layer, i.e., outside the area(s) with the apertures 704. Although the examples shown in FIGS. 7A-8B show seven sheets 702 included in the stack-up, in other examples other numbers of sheets 702 can be used.

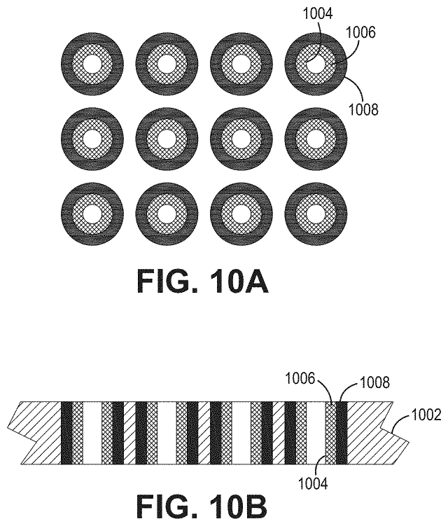

FIGS. 9A and 9B are a front view and a cross-sectional view of example steps in forming a main body 902 in which a filter is a monolithic structure with the remainder of the main body 902. In such an example, the main body 902 and filter can be composed of multiple sheets of polymer bonded together as discussed with respect to FIGS. 7A-8B. The filter portion(s) of the monolithic main body 902 defines airflow torture paths therethrough. Non-filter portions of the main body 902 can have solid surfaces, that is, non-filter portions do not define airflow torture paths therethrough. Such a main body 902 can be created by first forming a plurality of sheets of polymer having airflow path defining features therein as discussed above. One or more areas on the sheets of polymer can have airflow path defining features, while other areas can be impervious to air (i.e., solid surfaces with no path defining features). The sheets can be stacked and bonded such that the areas with airflow path defining features are generally aligned with corresponding areas on other sheets, yet still offset enough to create airflow torture paths as discussed above. This will create a stack-up (shown in FIG. 9A) of polymer having one or more areas with airflow torture paths defined therein and one or more other areas that are impervious to air. The stack-up can be thermo formed by applying heat and pressure represented by arrow 904 with appropriate tooling to achieve a desired geometry (shown in FIG. 9B) for the main body 902. In an example, more than half of the surface area of the main body 902 includes areas with airflow path defining features. In other examples, less than half of the surface area of the main body 902 includes areas with airflow path defining features. The heat and pressure to bond and form the desired geometry can be done in common steps or can be distinct steps. Advantageously, such a main body 902 can be sanitized and reused multiple times, because it is impervious to moisture.

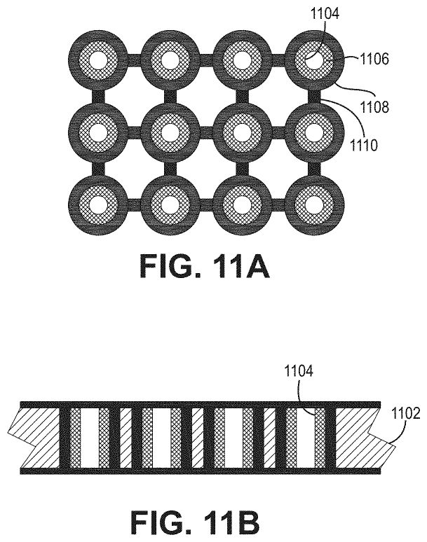

FIGS. 10A and 10B are front and cross-sectional views of another example sheet 1002 that can be used in a filter as described herein. The sheet 1002 includes apertures 1004 formed in accordance with any of the processes described herein. The apertures 1004 are plated with one or more metals to add anti-microbial benefits. The apertures can be plated using known metal plating techniques. In an example, the metal plating includes one or more metals and/or metal oxides selected from the group consisting of copper, silver, zinc, nickel, copper oxide, silver oxide, and zinc oxide. In the example, shown in FIGS. 10A and 10B, the apertures 1004 are plated first with copper 1006 and then silver 1008 is plated over the copper. In addition to providing anti-microbial benefits, plating with metal reduces the effective aperture size providing additional filtering capabilities to the airflow torture paths. One or more of such metallized sheets 1002 can be included in a stack of sheets to create airflow torture paths with anti-microbial benefits.

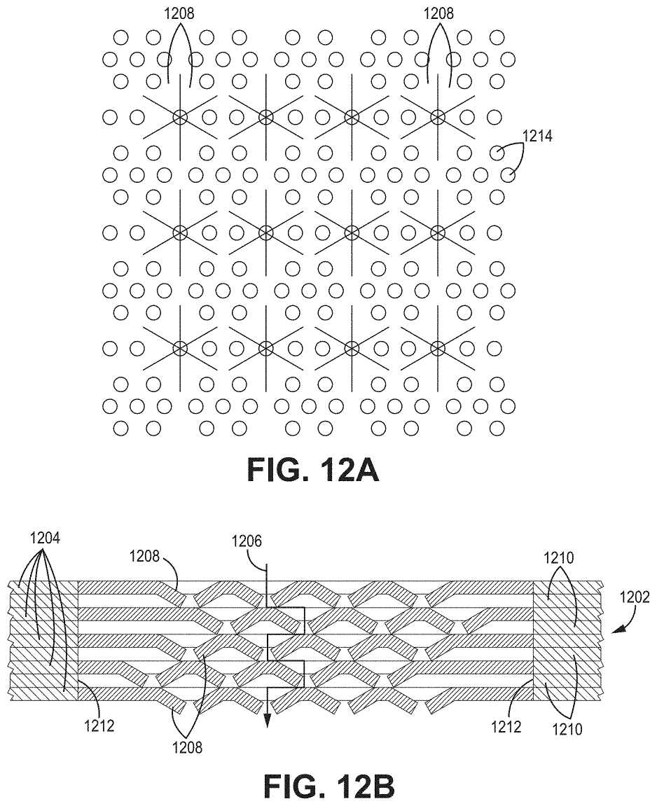

FIGS. 11A and 11B are front and cross-sectional views of yet another example sheet 1102 that can be used in a filter as described herein. The sheet 1102 includes metalized apertures 1104 that can be formed as described with respect to FIGS. 10A and 10B, including plating of copper 1106, silver 1108 and/or other metal as described above. Sheet 1102 also includes circuit traces 1110 (e.g., of copper) electrically coupling the plating in the apertures 1102 together to form a network of apertures 1102. This enables mass sterilization when the network of apertures 1102 are subjected to current sufficient enough to elevate the temperature of the metalized plating above the extermination temperature while below the melt temperature of the polymer sheet 1102. In some examples, the network of apertures 1102 can be electrically coupled to a power source such that electric charge can be applied to the network during use to enhance the lethal affect against the pathogens. In some examples, the circuit traces 1106 can be plated with silver on top of the copper to increase the surface area of anti-microbial regions with the mask.

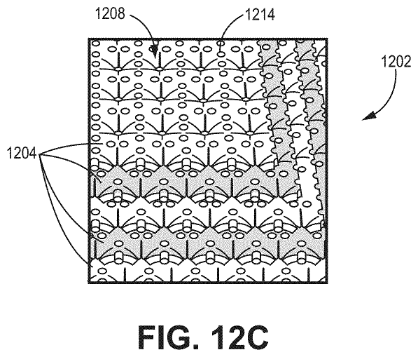

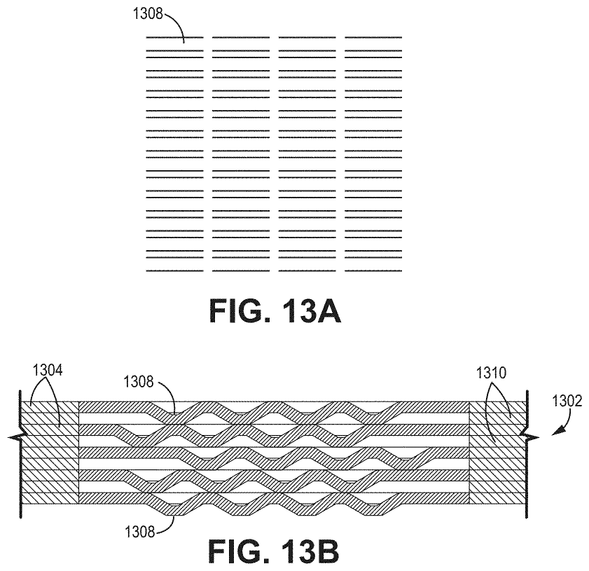

FIGS. 12A and 12B are front and cross-sectional views of another example stack-up 1202 of sheets 1204 defining airflow torture paths 1206 therethrough. In this example, the airflow path defining features in the sheets 1204 are flaps 1208. The flaps 1208 are sections of material that are configured to flex in response to pressure change during airflow exchange (i.e., human breath) through the airflow torture paths 1206. The sheets 1204 can be die cut or otherwise processed to create slits in desired locations to define the flaps 1208. In the example shown in FIGS. 12A and 12B, the flaps 1208 have a free hanging end that is free to move and are connected to the remainder of sheet 1204 via an opposite end. In this example, the flaps 1204 are triangular in shape defined by two slits on two sides of the triangle with the third side of the triangle connected. The triangular flaps 1208 can then flex along the third side enabling the flap 1208 to move (e.g., inward and/or outward) with respect to a base portion of the sheet 1204. In other examples, flaps with a free hanging end can have shapes other than triangular, such as rectangular.

As shown, multiple flaps 1208 can be defined in a sheet 1204 to provide multiple paths for airflow to traverse the sheet 1204. Multiple sheets 1204 can then be stacked to create the stack-up 1202 with airflow torture paths 1206 defined therethrough. A spacer sheet 1210 can be disposed between each sheet 1204 of flaps 1208 to provide space for the flaps 1208 to flex out of the plane of their sheet 1204. To provide that space, the spacer sheet 1210 can define one or more larger apertures 1212 that, for example, each span an entire area having multiple flaps 1208 or other airflow defining features thereon. A spacer sheet 1210 can be disposed between each sheet 1204 of flaps 1208 in the stack-up 1202. The sheets 1204 and flaps 1208 thereon can be disposed such that flaps 1208 in adjacent sheets 1204 are offset creating the airflow torture paths therethrough. In an example, other airflow defining features, such as apertures 1214, can also be defined in the sheets 1204. FIG. 12C is a cut-away view of the stack-up 1202 wherein the sheets 1204 are progressively cut-away to show the layering of the airflow defining features 1208, 1214. The sheets 1204, 1210 can be bonded together outside of the area(s) in which the airflow defining features are present. A bond layer can be used in some examples.

During use, as a human breathes with a facepiece on, the air pressure change and airflow through the filter can cause the flaps 1208 to flex inward and/or outward to allow air to pass through the stack-up 1202. In an example, one or both surfaces of the sheets 1202, at least in area(s) having flaps 1208 thereon, can have metal plating thereon to provide anti-microbial benefits. Including metal plating on the surface of the flaps 1204 causes air that travels through the flaps 1204 to be incident on the flaps 1204 when the flaps 1204 flex. Thus, air traveling along the airflow torture paths 1206 is forced into contact with the metal plating. The metal plating can then act to disable viruses and bacteria in the air as it travels between the interior cavity 106 and the external environment. In an example, the metal layer includes one or more metals and/or metal oxides selected from the group consisting of copper, silver, zinc, nickel, copper oxide, silver oxide, and zinc oxide. One or more of the sheets 1204 can have such a metal plating thereon.

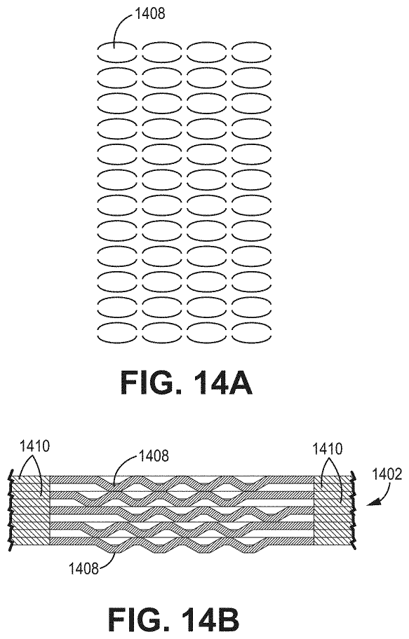

FIGS. 13A and 13B are front and cross-sectional views of yet another stack-up 1302. In this example, the stack-up 1302 defines flaps 1308 that are connected on two opposite ends thereof and operate by flexing (e.g., bending and/or twisting) in the middle between the two attached ends. In the example shown in FIGS. 13A and 13B, the flaps 1308 are defined by two parallel slits that form the flaps 1308 having a rectangular shape that is attached on opposite ends. In other examples, other shaped flaps can be formed. The sheets 1304 having flaps 1308 thereon can be stacked with spacer sheets 1310 therebetween to provide space for the flaps 1308 to flex. In an example, metal plating, such as that discussed above, can be included on one or both surfaces of one or more sheets 1304 of flaps 1308. The flaps 1308 can be disposed so they are offset from flaps 1308 on adjacent sheets 1304.

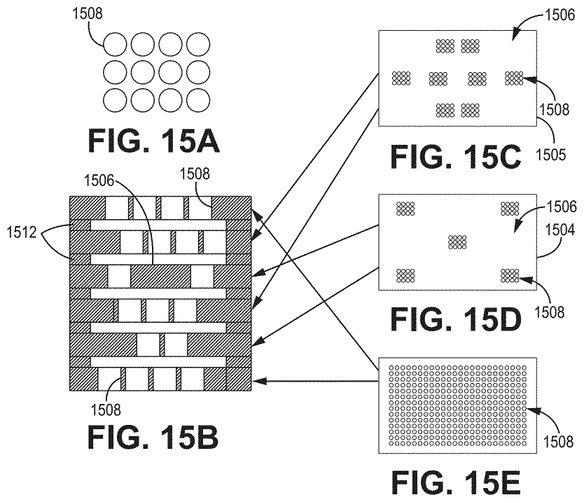

FIGS. 14A and 14B are front and cross-sectional view of yet another stack-up 1402. In this example, the stack-up 1402 defines flaps 1408 that are connected on two opposite ends thereof and operate by flexing (e.g., bending and/or twisting) in the middle between the two attached ends. In this example, the flaps 1408 have an oval (fish-scale) shape that is defined by two arced slits that arc away from one another. The sheets 1404 having flaps 1408 thereon can be stacked with spacer sheets 1410 therebetween to provide space for the flaps 1408 to flex. IN an example, metal plating, such as that discussed above, can be included on one or both surfaces of one or more sheets 1404 of flaps 1408. The flaps 1408 can be disposed so they are offset from flaps 1408 on adjacent sheets 1404.

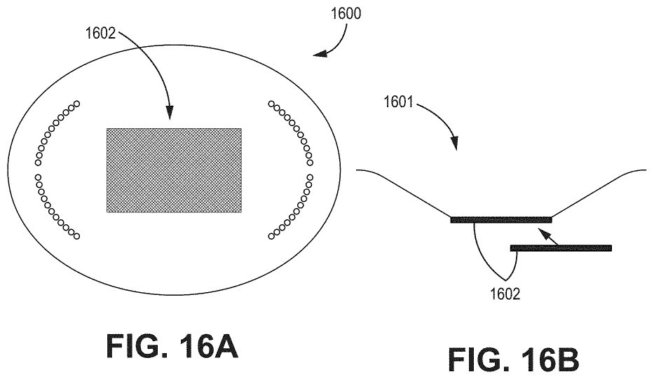

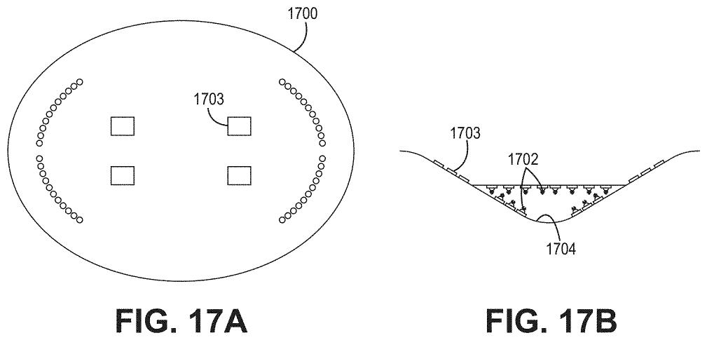

FIGS. 15A and 15B are front and partial cross-sectional views of still another stack-up 1502. In this example, sheets 1504, 1505 having portions that are impervious to air (e.g., solid surfaces 1506) are aligned with airflow defining features 1508 of adjacent sheets 1503, 1504, 1505 such that airflow through the airflow defining features 1508 is incident on the solid surfaces. The sheets 1504 having solid surfaces 1506 can have airflow defining features 1508 in other areas and the adjacent sheets can have solid surfaces 1506 in those other areas. Thus, as air traverses through the airflow defining features it is incident on the solid surfaces 1506. In an example, one or more of these solid surfaces can have metal plating thereon as discussed above to provide anti-microbial benefits. In an example, spacer sheets 1512 can be disposed between adjacent airflow defining feature sheets 1503, 1504, 1505 to provide space for air to travel horizontally past the solid surface to adjacent airflow defining features. FIGS. 15C, 15D, and 15E are front views of example sheets 1503, 1503, and 1505. In this example, outer sheets 1503 of the stack-up contain a large area of airflow defining features 1508 without integrated solid surfaces 1506. Internal sheets 1504, 1505 have areas of airflow defining features 1508 and other areas that are solid surface 1506. Internal sheets 1504, 1505 that are adjacent to one another have solid surfaces 1506 aligned with areas of airflow defining features 1508 of adjacent sheets 1505, 1506. The sheets 1504, 1512 can be bonded in areas outside the airflow defining features of all sheets 1504.

In any of the examples described herein with metal plating, the outer surface of the stack-up that is exposed to the inner cavity 106 of the facepiece can be free of metal plating to reduce the possibility of the metal plating flaking off and entering the breathable air stream. In an example, the airflow paths through the main body 102 can be sufficiently tortuous and small that pathogens are also captured (filtered out) as the air passes through the main body 102. For example, the airflow paths can capture sufficient pathogens that the facepiece 100 meets the N95 standard in force by the United States National Institute of Occupation Safety and Health (NIOSH) on Jan. 1, 2021.

Any of the features described herein can be mix and matched, with the basic principle of forming apertures, slits, or material separations that allow for air flow. The net airflow effect can be set by adjusting the pattern and/or size of slits, flaps, or apertures. In any of the examples described herein, the flaps can be less than 500 microns in length or less than 100 microns in length. In any of the examples described herein, the sheets can be less than 100 microns thick. The LCP can also be treated with a plasma deposited monomer to create anti-wetting characteristics where desired, as well as microfluidic channels can be added to control and direct fluid or moisture accumulation.

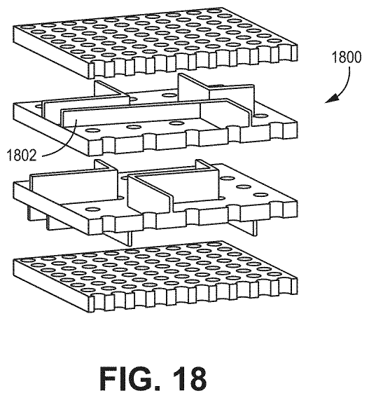

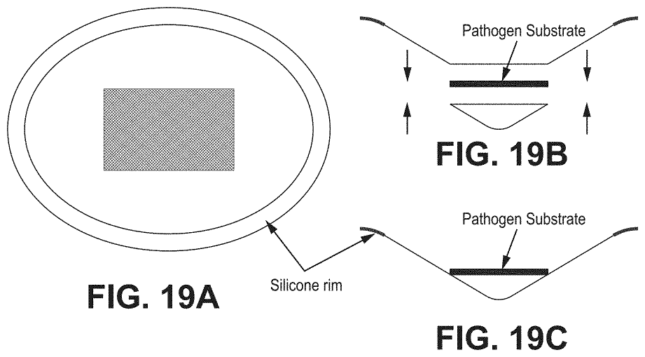

FIG. 16A is front view of an example facepiece 1600 having a filter 1604 formed in polymer sheets that are monolithic with the remainder of the main body 1602. FIG. 16B is a cross-sectional view of a facepiece 1610 having an exchangeable filter cartridge 1614 removably secured to a remainder of the main body 1612. The filter cartridge 1614 can include a filter having airflow torture paths as described in any of the examples herein. The main body 1612 to which the cartridge is removably secured can also be composed of one or more sheets of a thermoelastic polymer or can be composed of another material. Forming the filter 1614 in a cartridge 1612 enables the cartridge 1612 to be exchanged with a new cartridge as desired. The cartridge 1612 can be removed, replaced, cleaned, sanitized etc. Although an example size and geometry of the cartridge is shown in FIG. 16B, cartridge 1612 can have any suitable size or geometry. Example cartridges in which a filter can be formed and facepieces in which those cartridges can be used are provided in PCT patent application no. PCT/US21/29764 filed on Apr. 28, 2021 and titled “ANTIPATHOGEN RESPIRATOR”.

In an example, any of the torture paths described herein can be configured to achieve N95 type requirements for particle restriction. In an example, any of the torture paths described herein can have portions smaller than 5 microns across or smaller than 1 micron across. In an example, any of the stack-ups described herein can include three or more layers of airflow path defining features. For example, the stack-up can include three or more sheets of airflow path defining features having three or more layers of airflow path defining features in a given airflow torture path. In an example, any of the stack-ups described herein can include five, seven, or ten or more layers of airflow path defining features in a given airflow torture path. In any example described herein, airflow path defining features in a given area can be less than 100 microns apart from other features in their layer. In such examples where multiple areas of airflow path defining features are present, different areas can be more than 1 cm apart, wherein each area has features less than 100 microns apart from other features in their layer.

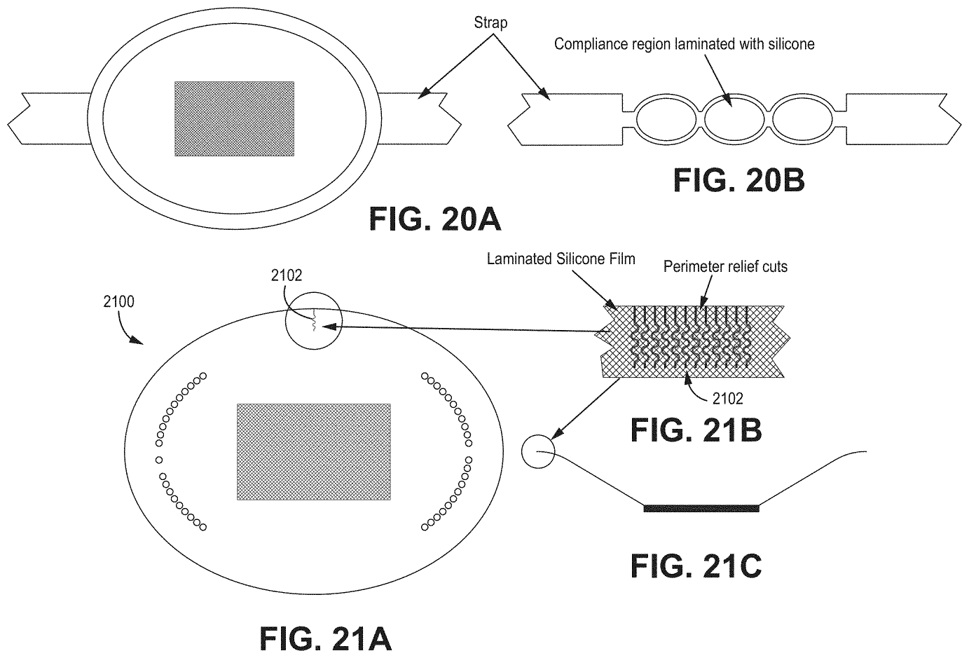

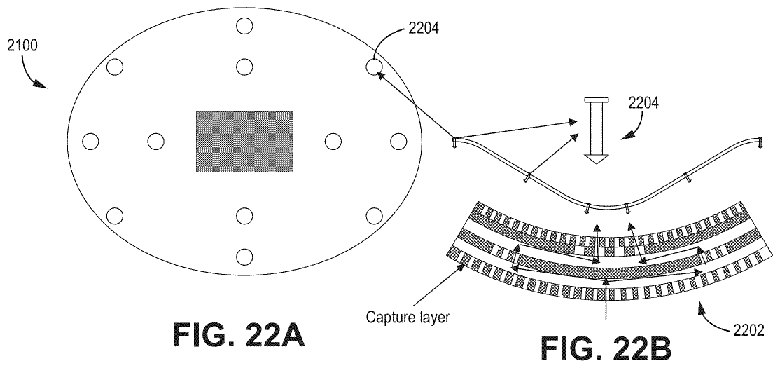

FIGS. 17A and 17B are a front view and a cross-sectional view of an example main body 1700 having embedded ultraviolet LEDs 1702 and other integrated circuit devices 1703 therein. Sheets of polymer can be disposed and bonded to define an air chamber 1704 through which the air flow through the respirator can be directed. One or more ultraviolet LEDs 1702 can be disposed to expose the air in the air chamber 1704 to UV light. Sufficient UV light can be provided to sanitize the incoming and outgoing air volume. Multiple integrated circuit and passive devices, including one or more sensors, processing devices, batteries, or transceivers can be embedded in the polymer layers to control power and provide wireless RF communication. One or more biological sensors can also be embedded in the polymer layers to perform diagnostic tests and identify presence or absence of pathogens, chemicals, gasses, etc. as well as notify need for cleaning or airflow restriction. The respirator 1700 can define airflow torture paths as describe herein between the air chamber 1704 and the interior cavity and/or the external environment. FIG. 18 is another example of air chambers 1802 that are created in a stack-up 1800. The air chambers 1802 are transverse passageways and can have UV LEDs or anti-viral surfaces through which the air flow is directed. Additional information regarding methods for embedding circuits, other components, and defining features with the polymer are disclosed in PCT Patent Application No. PCT/US2020/060631, filed on Nov. 15, 2020, and entitled “LIQUID CRYSTAL POLYMER EMBEDDED MICROELECTRONICS DEVICE”.

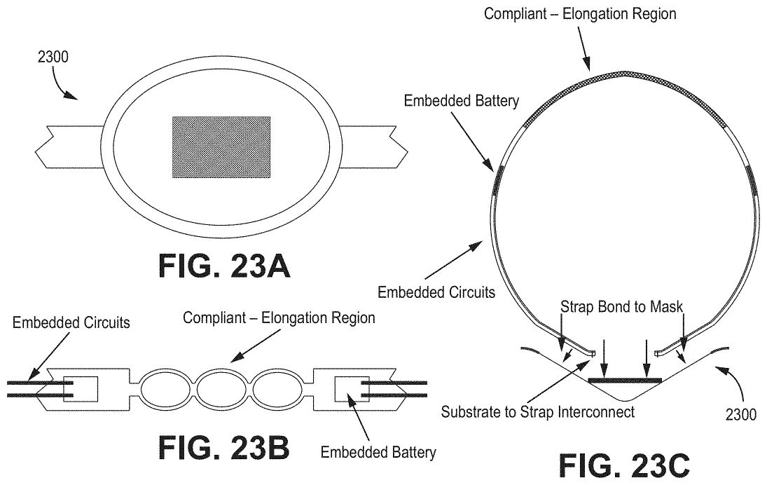

FIGS. 19A-C are a front view and two cross-sectional views of another example respirator having a cartridge with a stack of polymer layers/sheets as described herein. This respirator has a three main component construction where the main body that engages with the face is a thin injection molded part of appropriate size and shape to provide the best chance of accommodating a variety of face geometries, with a co-injected medical grade silicone rim to provide some compliance and conformal effect to improve sealing of the respirator and reducing airflow from around the perimeter of the respirator.

To create a mask assembly, the face frame serves as a mounting platform for the pathogen substrate, with a perforated cone mounted to the assembly to the complete mask. For the active UV LED version of the mask, the pathogen substrate can serve as the mounting platform for the LEDs and any related components.

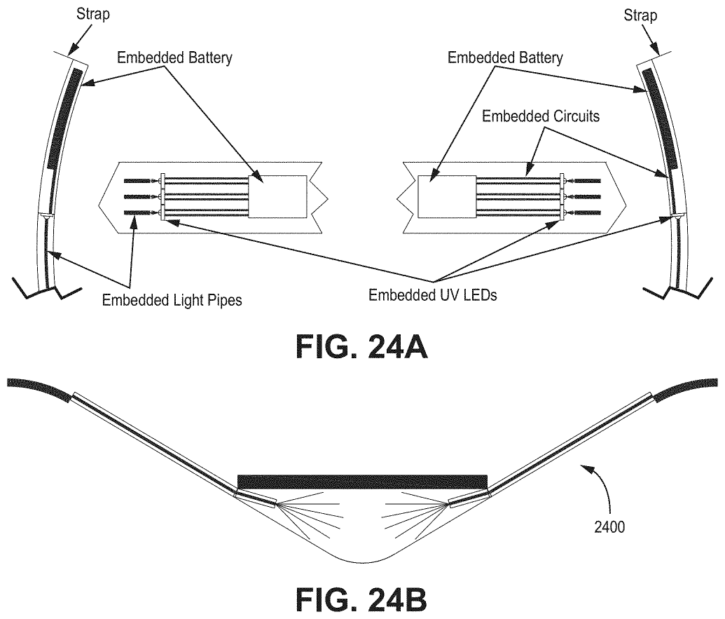

FIGS. 20A and 20B are front views of an example respirator having a polymer strap attached thereto. Strap design can be important to make sure the respirator is secured well and retained tight to the face while accommodating many user variables and remain washable or easily sterilized. The drawing below illustrates one option for a strap that is a strip of polymer that has features cut to provide the ability to stretch or elongate, with the feature region laminated with medical grade silicone to provide the elastic effect and prevent the thin features from being damaged. There may also be need for the strap ends at the mask interface to be adjustable, otherwise the basic strap can be fusion bonded to the face frame.

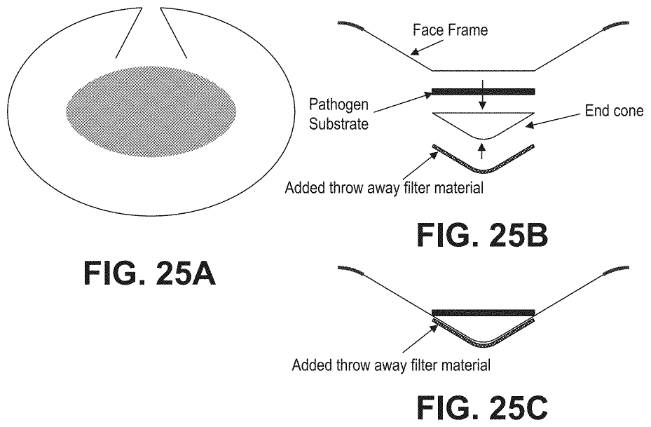

FIGS. 21A-C are a front view, an enlarged view, and a cross-sectional view of an example respirator 2100 having relief cuts 2102 defined therein. In an effort to improve the fit to the user's face, the perimeter of the polymer main body of the respirator 2100 can be patterned with relief cuts 2102 that allow for some level of compliance or form fitting. Silicone (e.g., a medical grade thin silicone layer) can be disposed over the relief cuts 2102 to contain the relieved polymer regions as well as provide the ability to stretch and conform to the user's facial structure and provide some level of sealing to drive airflow through the desired regions of the respirator 2100. Although only a single relief cut 2102 is shown in FIG. 21A it should be understood that a plurality of relief cuts 2102 can be defined in the perimeter of the main body as desired.

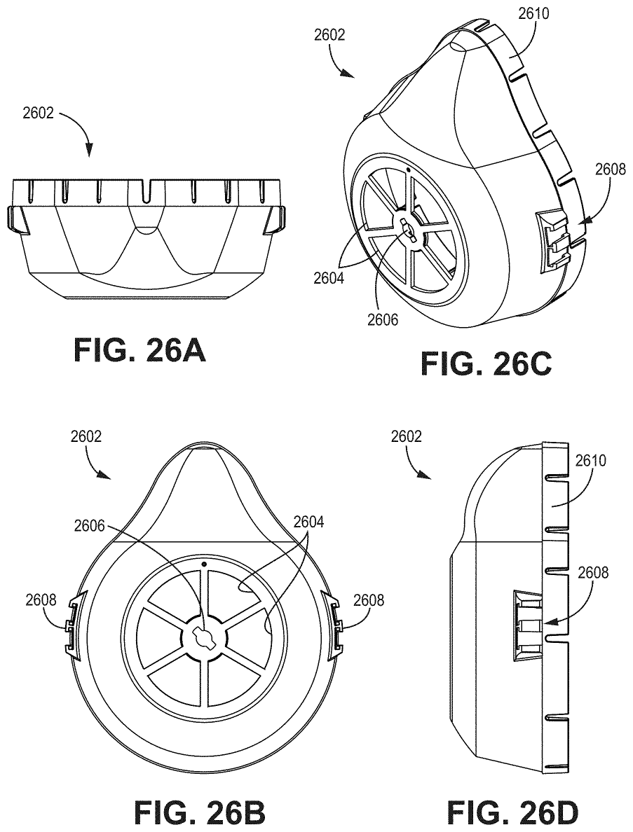

FIGS. 22A and 22B are a front view and a cross-sectional view of an example off-the-shelf respirator 2200 having a cartridge formed of a plurality of polymer layers as described herein attached thereto. In an example, a cartridge as described herein can be configured to attached to an off-the-shelf respirator to enhance the filtering ability of the respirator and/or add additional functionality to the respirator, such as antiviral properties. A thin pathogen cartridge 2202 that is either conformal or formed to shape and attached to the inner surface of the off-the-shelf respirator 2200. Hooks, or prongs or pins 2204 can be used to affix the pathogen shield to the fabric of the existing mask with the intent of retention during use but also removal and placement on another mask after cleaning and sanitation. In an effort to keep complexity and cost down, a simple torture path of only a few layers in the cartridge 2202 is likely adequate to improve the pathogen disabling effect while the existing mask provides a level of filtering. A thin capture layer on the outer surfaces of the pathogen layers can prevent direct contact with the silver plating.

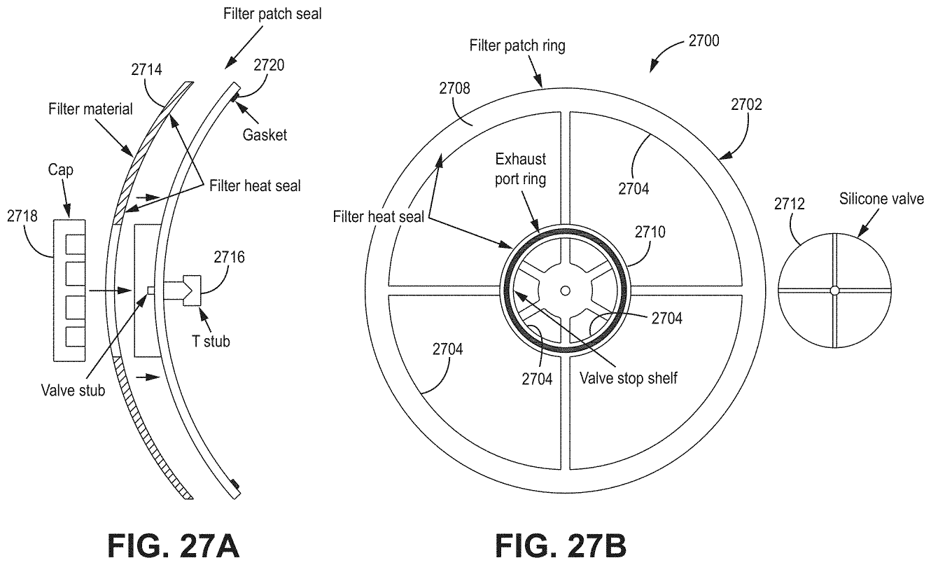

FIGS. 23A-C are front views and a cross-sectional view of an example respirator 2300 having a strap with embedded circuits therein. A significant advantage is the ability to embed and power Ultraviolet LEDs within the mask structure. Studies have shown that 2 mJ/cm-squared of 22 nm wavelength UV light is effective in disabling viruses in airborne form. In an example, this respirator 2300 is configured to provide a UV source and UV power supply within the structure of the mask and the strap affixing the mask to the head and face such that during use the user's airstream is exposed to the UV pathogen disabling effects. The mask can be constructed with the UV feature as a stand-alone version or combined with the anti-pathogen silver bearing matrix.

Respirator 2300 includes an embedded solid state or thin film polymer batteries within the mask strap, as well as potentially any power management or sensor devices as needed. Embedded circuits extend to an interconnect point to the pathogen substrate which can be a stud bump, or solder joint, or connector etc. In some cases, the strap may be separable from the mask, while in some cases it may be desirable to permanently bond the strap to the mask. In some cases, it may be desirable to replace the batteries, and in some cases the batteries are to be embedded with appropriate charging circuitry and electronics to accommodate wireless charging, or direct interconnect to a charging mechanism either while connected to the mask or discretely when detached from the mask. This electronic function can be used when UV LEDs are contained within the mask structure, or there may be of benefit to provide current or charge to the pathogen matrix during use or during sterilization to increase the efficacy of disabling pathogens.

The above example relies upon the premise that UV LEDs are located within the air chamber, likely mounted to the pathogen substrate or potentially the interior of the mask itself. It is important to position the LEDs in proper direction and in sufficient quantity such that the light shroud or effective impact are covers the majority of the effective region within the chamber and path of the airflow. In an example, the light shroud can be contained and restricted to the air chamber as much as possible as to prevent UV leakage beyond the desired chamber whether exposing the wearer or the area outside of the immediate mask areas. From an electronics efficiency point of view, the LEDs and power management functions can be disposed as close to the power source as possible. The precision embedded circuits do provide a very low resistance path, and the active LEDs within the chamber will likely mounted in a way that can help manage any thermal or heat generation issues.

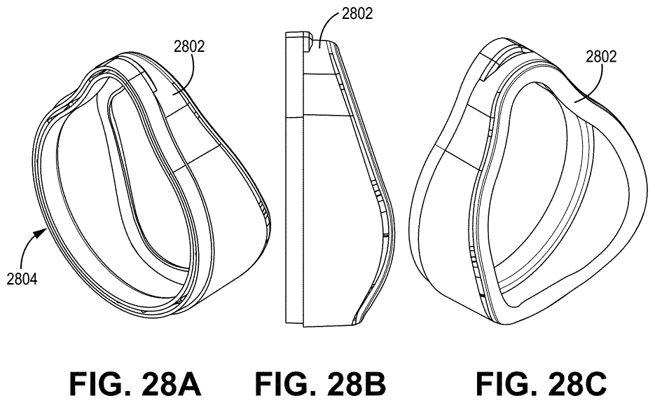

FIGS. 24A and 24B are cross-sectional views of yet another example respirator 2400 having batteries embedded in the straps and light pipes for delivering the light to the air chamber. This is an alternative or addition to the air chamber mounting of the LEDs. Here the LEDs are embedded within the strap structure and create a light pipe that transmits UV light into the air chamber at proper locations with the light transmission aimed at the appropriate air chamber areas. The battery power is embedded within the strap architecture, and also embedding the UV LEDs within the strap in such a way that light pipes are aligned to the LED emitter and transmit the UV light through the strap and aligned to the air chamber to saturate the cavity with UV light to disable pathogens within the airstream.

Additional information regarding methods for embedding circuits, other components, and defining features with the polymer are disclosed in PCT Patent Application No. PCT/US2020/060631, filed on Nov. 15, 2020, and entitled “LIQUID CRYSTAL POLYMER EMBEDDED MICROELECTRONICS DEVICE”.

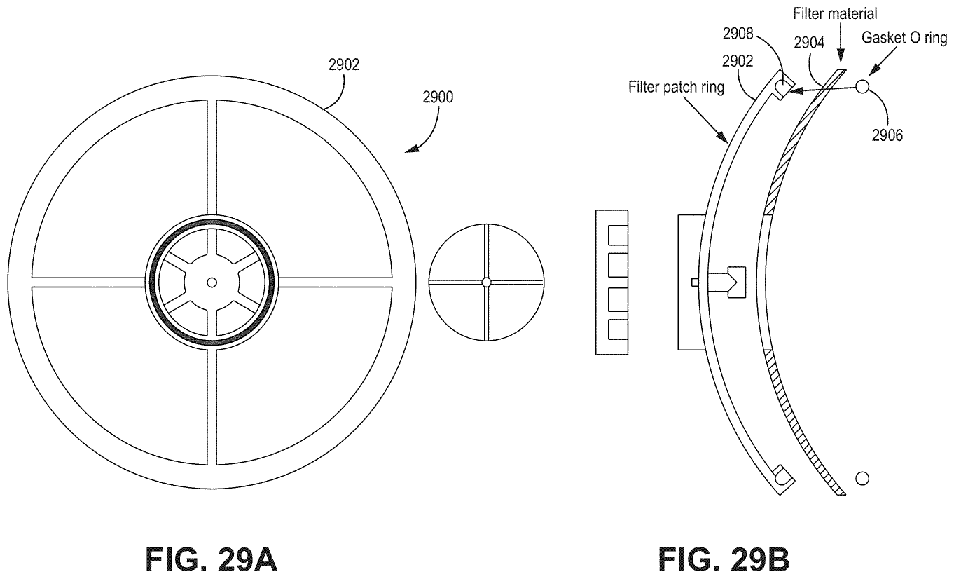

FIGS. 25A-C are front views and cross-sectional views of a respirator 2500 that combines the filter effects of a traditional N95 or KN95 type facial mask and the benefits of the stacked polymer layers as described herein. In this example, a combination mask is shown where a construction consists of a face frame with a sealing gasket flange, and an end cone that provides structural support for a N95 type filter material such as blown fiber matting, with a stacked polymer layer defining tortuous air passages mounted near the interface of the face frame and the end cone.

The benefits of the stacked polymer concepts over conventional protective facial masks can include: 1) The polymer construction creates a protective facial mask that can be cleaned, sterilized, and reused many times. 2) The polymer construction can be processed with plasma deposited monomer to enhance non-wetting properties. 3) The polymer construction can be fabricated in a simple passive form as a filter similar to conventional masks. 4) The polymer construction can be enhanced to provide active pathogen attacking features and function which is absent from conventional facial masks. 5) A metalized network can be connected to current for sterilization or thermal or electrical treatment to enhance pathogen destruction. 6) The polymer construction can be fabricated to add electronic function to enhance the pathogen attacking effectiveness, as well as embedded sensors to monitor the environment or contact with pathogens, wireless communication to report conditions and data, power management and charging functions to facilitate solid state battery power.

Conventional N95 respirators are typically constructed of layers of filtration materials commonly referred to as non-woven, or melt blown non-woven. This material as the name implies, relies on relatively random yet massive amounts of polymer filaments that when meshed together create a filtration effect of spider web like structures intended to capture small particles as they pass through the matrix. To enhance the filtration efficiency, the material is typically statically charged such that when particles enter the matrix the static electricity improves the capture percentage.

A key aspect of the N95 respirator products is they must be fit properly to the face in order to achieve desired target filtration. If not properly fit, the airflow tends to escape and enter around the perimeter of the respirator which defeats the filtration principle with unfiltered air entering and exiting. A requirement for proper fitting is adequate training of the user to properly fit the respirator to their particular facial structure to avoid leakage. In general, this training is done on a yearly basis with the use of a fit check tester that measures the pressure drop and leakage potentials. This test is not done every time the user wears a respirator, and the effectiveness relies upon the user's skill and diligence to achieve proper fit to maximize filtration.

The harsh reality of the this fit to face requirement is the material has some compliance but must be held to the face with significant pressure which essentially relies partially on the compliance of the face itself. In some cases, a silicone rim is over-molded to add some compliance. The retention mechanism that holds the respirator against the face is typically behind the ear or behind the head loop elastic straps that are attached to the respirator at roughly the 2 and 4 and 8 and 10 o'clock positions. The requirement for proper no leak fitting makes the respirator difficult to wear comfortably for long periods of time and often results in skin issues at the interface locations.

The nature of the filtration mechanism also significantly increases airflow restriction which is a balancing act between particle capture and ease of breathing. In some cases, one way exit valves are used to relieve the exhalation pressure to ease breathing but these structures do not filter the outgoing airflow and have been avoided. These respirators are also intended to be a one-time use and discarded, with a fresh replacement at each sequential use. The nature of the filtration material capturing particles, retaining moisture and contamination also make the respirators impossible to clean and very difficult to sanitize. Use of a contaminated respirator can dramatically increase the risk of infection to the user or those nearby. Another short coming is the respirator obscures and covers the users face while also muffling conversation, understanding and facial expression.

Another shortcoming of the filtration principle is a significant percentage of the exhaled air is rebreathed during the subsequent inhalation. If the user is exhaling pathogens, then they will continually infect themselves by rebreathing expelled air as well as wearing a contaminated respirator.

Although N95 respirators are very difficult to wear, must be fit properly to provide proper protection, and are impossible to clean they are the recommended best protection against airborne pathogens and do provide some protection from surface pathogen transmission by keeping the user from touching the face.

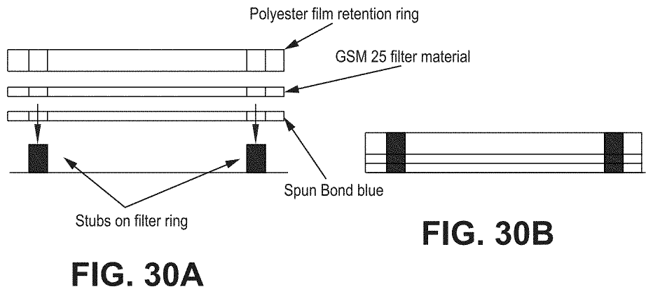

FIGS. 26A-D are perspective views of an example base frame 2602 of a main body to which a cartridge can be removably secured. The base frame 2602 and cartridge are configured such that the cartridge can be removably secured to the base frame. That is, the cartridge can be secured to the base frame 2602 for use and, after some length of use corresponding to a lifetime of the cartridge, the cartridge can be removed from the base frame 2602 and cleaned or discarded. A new or cleaned cartridge can then be re-secured to the base frame 2602 to continue using the respirator. The base frame 2602 can be configured to block air flow between an internal cavity and the external environment, except for where the cartridge is secured thereto. The base frame 2602 can define one or more apertures 2604 where the cartridge is attached thereby directing the airflow between the internal cavity and the external environment through the cartridge. In this example, the apertures 2604 are disposed proximate a mouth of the user, however, in other example, apertures can be disposed in other areas instead of or in addition to apertures 2604. The base frame 2602 can define a slot 2606 configured to accept and mate with a T stub on a cartridge as described below.

The base frame 2602 can also define one or more strap connectors 2608 for connection of one or more straps for securing the base frame 2602 onto a head of a user. In this example there is a strap connector 2608 on the exterior of each lateral side of the base frame 2602. The base frame 2602 can also define an edge region proximate a face of the user having a profile configured to have a silicone or other compliant material disposed thereon for compliant and comfortable contact between the base frame 2602 and a user's face.

Such a design with a cartridge secured thereto provides a respirator that has a reusable base frame 2602 that can be easily cleaned and sanitized hundreds if not thousands of times. The respirator can be easy fit to face with comfortable sealing and no skin irritation issues. The respirator can provide self-adjustment retention to provide proper seal with minimal pressure against the face. The respirator 2600 can provide targeted filtration to optimize airflow and comply with N95 rating standards. The respirator can have easily replaceable filtration via the cartridge with multiple cartridge options considering environment and protective needs. The base frame 2602 of the respirator can be composed of an optically clear material to allow for visual viewing of the wearers face and expressions. The respirator 2600 can add pathogen destruction and disablement beyond simple filtration.

The base frame 2602 can be constructed from a wide variety of polymer choices that can be molded, shaped or thermo-formed such as Liquid Crystal Polymer, Polycarbonate, PEI, Acrylic, Silicone, Neoprene etc. The physical properties of the base frame can vary widely depending on desired features and functions such as rigidity, weight, transparency, flexibility, etc.

In one example, base frame 2602 has a construction based upon Polycarbonate or Acrylic polymers that provide mechanical infrastructure to incorporate multiple components and features while being optically clear to allow for the users face and facial expressions to be viewed while conventional respirators block the view of the face. These polymers will typically yield a rigid structure but depending on the design the respirator could be made flexible or partially flexible for reasons such as flat storage or distribution.

From a product acceptance standpoint, the general shape and appearance of the base frame 2602 and overall respirator is generally curvilinear and in basic terms serves as the structure that holds and presents the filtration and anti-pathogen structure at the proper location within the airflow path. The structure also serves as the skeleton for arranging and attaching various components that provide features such as facial sealing, respirator retention on the user's head, filter replacement, electronics integration, filter attachment etc.

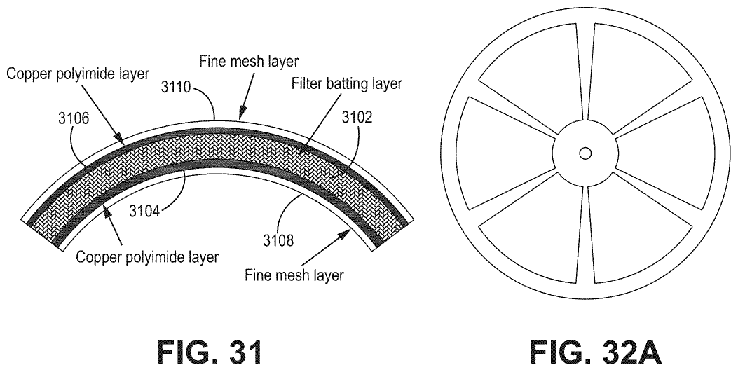

FIGS. 27A and 27B are exploded views of an example cartridge 2700 for use in any of the cartridge respirators described herein, including with base frame 2602. As shown in FIG. 27A, the cartridge 2700 includes a molded support member 2702 that defines one or more inlet apertures 2704 and one or more outlet apertures 2706. In this example, the support member 2702 defines an outer ring 2708 and an inner ring 2710. The one or more inlet apertures 2704 are defined between the outer ring 2708 and the inner ring 2710. The one or more outlet apertures 2706 are defined inside the inner ring 2710. The inner ring 2710 therefore is also referred to herein as an exhaust valve ring. The support member 2702 defines a shallow tube feature that extends from the inner ring 2710 and functions as the exhaust port. This exhaust port is located in the center of the support member 2702 such that it is positioned immediately in front of a user's mouth while being worn. Accordingly, when a user exhales, the general direction of the exhale does not have to change, ensuring there is a high force on the exhaust valve and a high level of the exhaling breath leaves the respirator quickly. The cartridge 2700 includes an exhaust valve 2712 that is disposed in the exhaust port and restricts air coming into the interior cavity of the respirator while allowing air to easily exit from the interior cavity of the respirator. In this example, the exhaust valve 2712 is a silicone valve having a shape that covers substantially all of the one or more outlet apertures 2706. The exhaust valve 2712 is disposed on the outward side of the one or more outlet apertures 2706 and extends all the way over the apertures 2706 to the structures on all sides of the apertures, such as to a valve stop shelf defined on the outside edges of the apertures and the internal spokes extending from the valve stop shelf to an internal hub of the support member 2702. In an example, the outward facing surfaces of the valve stop shelf and internal spokes are at a common position such that the silicon valve when forced inward during inhale contacts the valve stop shelf and internal spokes at the same time and therefore seals the outlet apertures 2706. Conversely, a cap 2718 that covers the silicone valve in the exhaust port provides a cavity on the outward side of the silicone valve allowing the silicone valve to flex and allow air flowing out of the outlet apertures 2706 to exit the respirator. The exhaust port has a post is located in the center to locate and affix the valve 2712. The valve 2712 itself is a thin molded silicon sheet with a hole in the center for the post, and some raised ribs to help bias the valve against the valve stop shelf which is interior to the exhaust port ring.

A filter material 2714, such as a N95 type filter material, is disposed over the inlet apertures 2704 to filter incoming air entering the respirator. In this example, the filter material 2714 has a generally annular shape and extends from the inner ring 2710 to the outer ring 2708. The filter material 2714 is cut to a diameter shape, with a hole in the center to clear the exhaust port tube. The material is heat sealed to a shelf that is exterior to the exhaust port as well as the outer perimeter of the ring to seal and prevent leakage. A molded cap 2718 is included that slips onto the outer diameter of the exhaust port ring 2710, with slots around the perimeter wall that allow for the exhaust air to exit freely. The interior of the support ring is shown with 4 thin webs to maximize air flow area and provide some theoretical compliance around the ring when loaded against the gasket-mask face seal.

The cartridge also defines a cartridge securing member that is configured to removably secure the cartridge to the base frame 2602. In an example, the cartridge securing member is a physical structure that removably interlocks with a corresponding structure on the base frame 2602 of the facepiece. In other examples, other cartridge securing members can be used, such as an adhesive, magnet, or Velcro type feature. In this example, the cartridge securing member is a ‘T’ stub feature 2716 extending therefrom that mates with a corresponding female slot 2606 on the base frame 2602. The T stub 2716 and corresponding slot 2602 are configured such that the cartridge can be removably secured to the base frame 2602 by inserting a T stub 2716 into the slot 2602 and rotating the cartridge relative to the base frame 2602 to lock the taps of the T stub 2716 into the slot 2606. A gasket 2720 is located around the perimeter of the support ring to seal against the base frame 2602 surface during the twist action that is intended to pull the ring towards the surface of the base frame 2602.

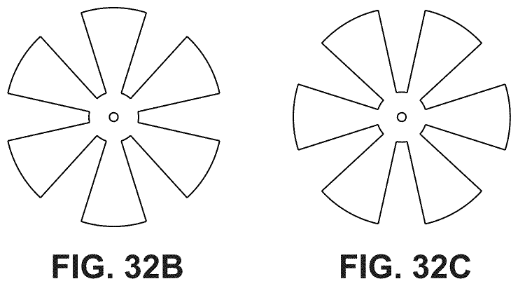

FIGS. 28A-C are perspective views of an example compliant face rim 2802 that can be attached to the base frame 2602 shown in FIGS. 26A-D. Existing respirators use an over-molded silicone rim to improve sealing in lieu of forcing the base respirator material to conform or seal against the face with great pressure and discomfort. In contrast, the rim 2802 shown in FIGS. 28A-C is detachable, such that multiple size rims 2802 can be affixed to a common base frame 2602. In an example the rim 2802 is composed of silicone and defines a slot 2804 that is stretches around tabs 2610 on the base frame 2602 to detachably connect the rim 2802 to the base frame 2602. This enables removal of the rim 2802 for sterilization and selection of a rim 2802 that is best configured to the shape of the user's face. Thus, an easier means of achieving a good fit is provided.

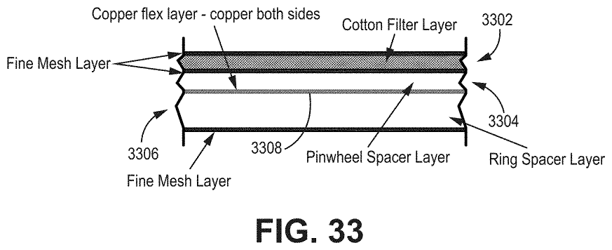

FIGS. 29A and 29B are a front view and a cross-sectional view of another example of a cartridge 2900. This is similar to cartridge 2700 except the filter material 2904 is disposed internally to the support member 2902. The filter material 2904 is affixed with an O ring style gasket 2906 that secures the filter material in place by pinching the material 2904 into a groove 2908 defined in the support member 2902. The gasket 2906 also provides the seal to the main body of the respirator. This example can provide touch points on the support member 2902 for the user to use during securing to the main body. These touch points allow the user to twist against the ribs rather than touching and potentially contaminating the filter material before use.

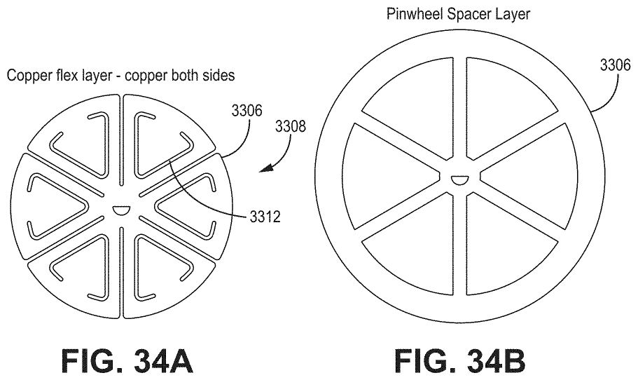

FIGS. 30A and 30B are cross-sectional views of an example of a cloth stack 3000 that can be used as filter material on a cartridge as described herein. The cloth stack 3000 includes a polyester film retention ring 3002 and a layer of filter material 3004 (e.g., a GSM 25 material) disposed underneath the polyester film. A spun bond glue 3006 is disposed under the filter material 3004 and a plurality of stubs 3008 extending from a support member of a cartridge are used to secure the retention ring 3002 and filter material 3004 onto the support member.

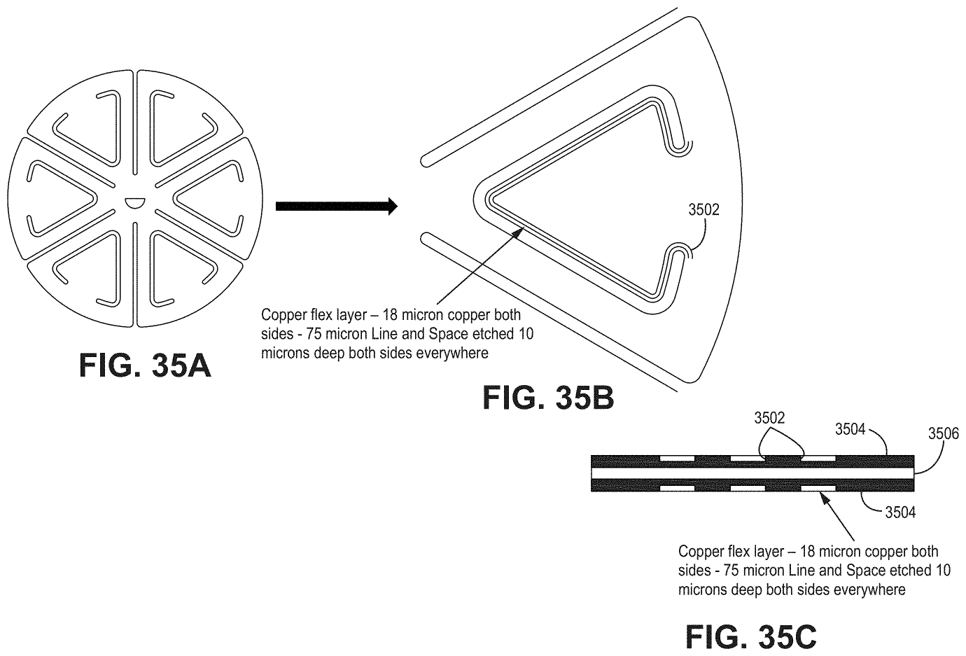

FIG. 31 is a cross-sectional view of a filtering portion that can be used in a cartridge or in a main body of a respirator. The filtering portion can include a filter batting layer 3102 that functions as a filter material and has a layer 3104, 3106 of polymer with copper thereon on both sides of the batting layer. The polymer-copper layers 3104, 3106 can be secured to the batting layer 3102 and can define a plurality of spaces for air to flow therethrough. In an example, the spaces in polymer-copper layer 3104 on one side can be alternated with the spaces of the polymer-copper layer 3106 on the other side such that the air is directed into contact with one or both of the layers 3104, 3106 as it passes through the cartridge. A fine mesh layers 3108, 3110 can be disposed on the external sides of the polymer-copper layers 3104, 3106.

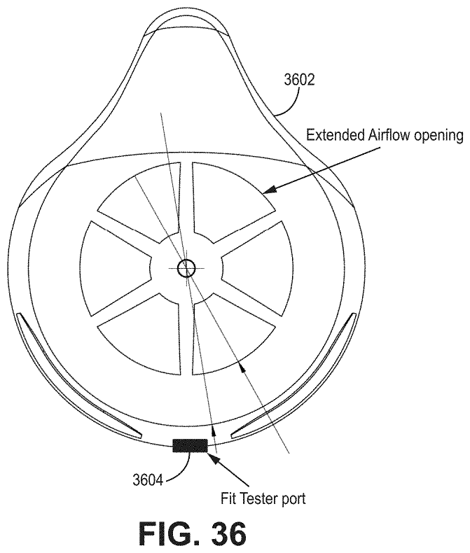

FIGS. 32A-C are front views of example components for the filtering portion of FIG. 31. FIG. 32A is an example support member to which the filtering portion can be attached. The support member includes a plurality of apertures that function as both inlet and outlet apertures for the respirator. The support member can include a member to removably secure the cartridge to a main body, such as T stub located in a center thereof. In an example, the support member has a circular shape with an outer ring, an inner hub, and spokes extending from the outer ring to the inner hub. The spaces defined between the outer ring, inner ring, and spokes comprise the inlet/outlet apertures are described herein. FIGS. 32B and 32C are front views of example polymer-copper layers showing the relative orientation of each such that air flow is directed past each of them.

For the copper polyimide layers, the idea is to have a cut shape that provides copper both sides with surface areas that drive airflow against the copper relative to the webs in the molded cone on the mask. The drawing below is very crude to illustrate the principle, with the left image being the inner layer corresponding to the web configuration on the molded mask and the right image is a corresponding rotation in the pattern to try and put copper in the way of the air flow as it passes through the batting material into the user inhale and be in the way of exhale airflow as much as possible considering the relief valves. The spacer gap created by the batting layer is hoped to provide enough room for air to flow through the batting filter material and come into contact with the outer and inner copper layers.

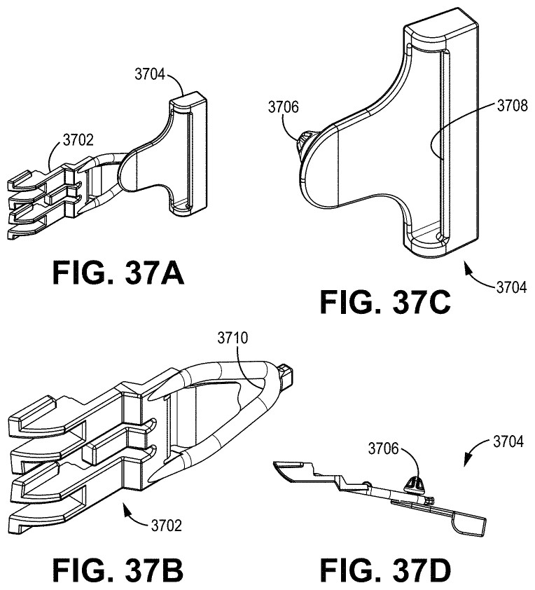

FIG. 33 is a cross-sectional view of another example filtering portion that can be used in a cartridge or in a main body of a respirator. The filtering portion includes a filter layer 3302. In an example, the filter layer 3302 is composed of cotton. The filtering portion includes a ring spacer layer 3304 with a fine mesh layer between the ring spacer layer 3304 and the filter layer 3302. The filtering portion also includes a pinwheel spacer layer 3306 and a copper flex layer 3308 disposed between the ring spacer layer 3304 and the pinwheel spacer layer 3306.

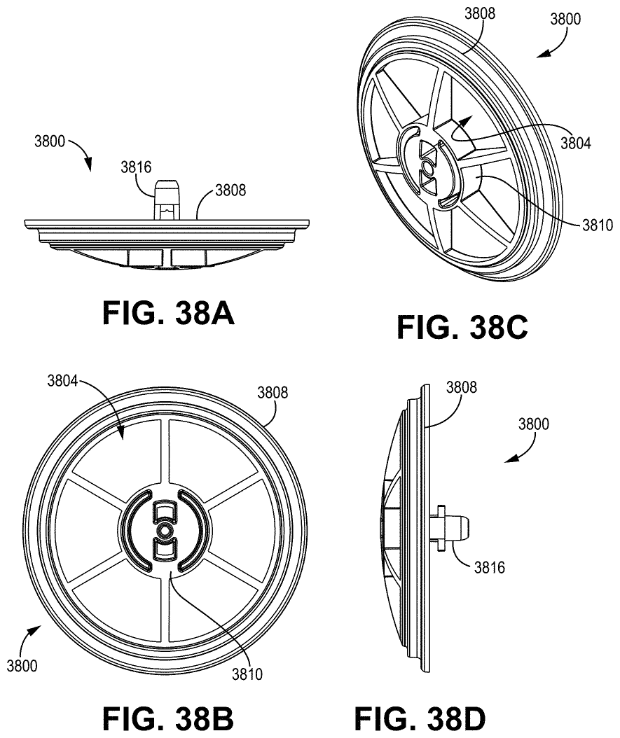

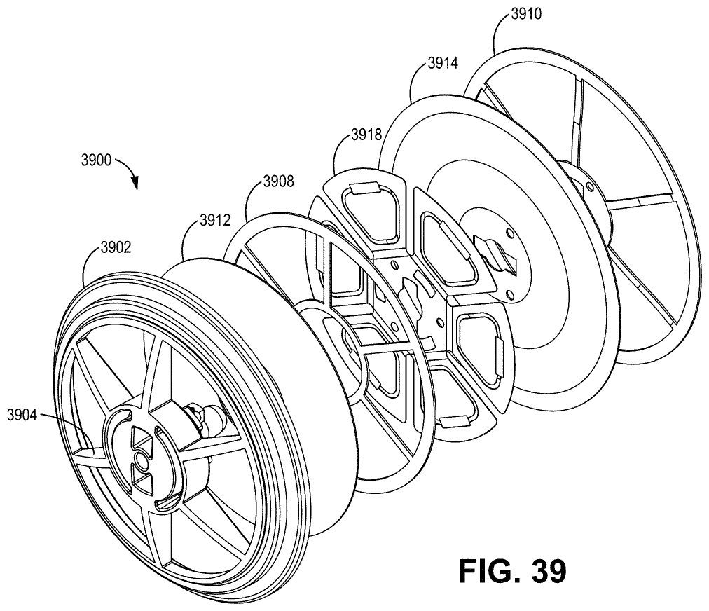

FIG. 34A is a front view of the copper flex layer 3308 and FIG. 34B is a front view of the pinwheel spacer layer 3306. Another fine mesh layer can be disposed on the outside of the cotton filter layer 3302 and on the outside of the pinwheel spacer 3306. FIG. 34C is a side cross-sectional view of the filter portion of FIG. 33 illustrating its concave shape to provide natural space for a user's face.

The copper flex layer 3308 can be composed of a polymer layer having a copper layer on both sides of the polymer layer. The copper flex layer 3308 defines a plurality of flaps that are configured to flex slightly in response to air flow into and/or out of the respirator. The flex in the flaps allows for air to more easily flow through the copper flex layer 3308. The flex, however, is kept low (e.g., less than 30 degrees from normal) such that the air flows past the angled flaps as it travels through the copper-flex layer 3308. In an example, the one or more flaps are configured to flex both inward and outward to allow air to flow both inward and outward past the flaps. In another example, a first one or more flaps are configured to flex inward for incoming air and a second one or more flaps are configured to flex outward for outgoing air.

The copper flex layer 3308 is configured to contact the pinwheel spacer 3306 shown in FIG. 34B. The pinwheel spacer 3302 includes an outer ring that contacts an outer edge of a first plurality of larger flaps 3310 defined in the copper flex layer 3308. The pinwheel spacer 3302 also includes a plurality of spokes that extend from the outer ring and contact the larger flaps 3310 along respective sides thereof. The contact between the pinwheel spacer and the larger flaps 3310 prevents the larger flaps 3310 from flexing towards the pinwheel spacer 3306. In an example, the copper flex layer 3308 is secured to the pinwheel spacer 3306 proximate a center thereof. The ring spacer 3304 has an outer ring without spokes. The outer ring is disposed outward of the larger flaps 3310, such that the larger flaps 3310 can flex into the large aperture formed by inside the outer ring. In this way, the filtering portion enables the larger flaps 3310 to flex one way and restricts them from flexing the other way.

The copper flex layer 3308 also defines a plurality of smaller flaps 3312. In this example the smaller flaps 3312 are defined within the larger flaps 3310. The pinwheel spacer 3306 such that it does not contact the smaller flaps 3312, thereby allowing the smaller flaps to flex towards the pinwheel spacer. In this way, the larger flaps 3310 are configured to flex one direction (e.g., outward) and the smaller flaps 3312 are configured to flex in the other direction (e.g., inward). Advantageously, by embedding the smaller flaps 3312 in the larger flaps 3310, the surface area of surface area of the copper flex layer 3308 is well utilized. This is because air is forced against a large portion of the first side of the copper flex layer 3308 when the larger flaps 3310 flex in the first direction. Additionally, air is forced past a large portion of the second side of the copper flex layer 3308, reverse of the first side when the smaller flaps 3312 flex in the second direction. In other examples, the copper flex layer can have other antiviral materials in addition to or instead of copper, such as silver and/or zinc. In yet other examples, the copper flex layer 3308 can be composed solely of a metal such as copper, silver, or zinc (e.g., at least 90%, 96%, or 99.9% pure copper, silver, and/or zinc).

Accordingly, the copper flex layer 3308 uses both sides as an anti-pathogen layer as well as a pseudo exhaust valve contained within the filter. In an example, the fine mesh layer on the outside of the batting layer 3302 is a thin cotton outer layer to contain any filter layer fibers and provide a clean outer surface. In an example, the batting layer 3304 is a cotton batting filter layer or alternate N95 type spun polyester non-woven filter material. In an example, fine mesh cotton between the ring spacer 3306 and the batting layer 3304 contains the filter batting material and keeps the batting filter material spaced from the copper flex layer 3308 to allow space for the flaps of the copper flex layer 308 to flex. In an example, the copper flex layer 3308 is composed of a polymer, such as Kapton, polyester, polyolefin, LC with copper on both sides. In an example, the slots defining the flaps in the copper flex layer 3308 are 1 mm across. In an example, the pinwheel spacer layer 3306 matches the webs on the main body and allows the major (larger) flaps 3310 of the flex layer 3308 to flex outward during exhale and allows the interior (smaller) arrowhead shaped flex flaps 3312 to flex inward during inhale. The pinwheel spacer 3306 can be a molded part to provide contour support and keep the cotton batting filter layer from bunching or trying to impede the flex features from moving. In an example, the fine mesh layer on the outside of the pinwheel spacer 3306 can enclose the copper flex features. In an example, a ring of PSA will go around the periphery of the filter about 5 mm wide and can be placed on the pinwheel spacer 3306 if the cotton layer does not extend all the way out to the edge.

As described above, the major flex flaps 3310 of the copper flex layer 3306 resides on the webs of the pinwheel spacer 3306 which will prevent inward flexure during inhale, while the spacer ring 3304 provides space for the major flaps 3310 to flex during exhale. The interior flex flaps 3312 can flex inward during inhale and will likely be subordinate during exhale. The goal is to have the airstream flow through the filter batting layer inhale and exhale while driving the airflow in contact with the exposed copper as much as possible without restricting too much airflow without large perforations. The goal is also to provide the copper pathogen effect in a single circuit layer. Another copper circuit layer (e.g., copper flex layer 3308) can be added to increase the anti-pathogen effect.

Although a specific geometry for the flaps of the copper flex layer 3308 is shown and described, other geometries having flexible flaps can be used.

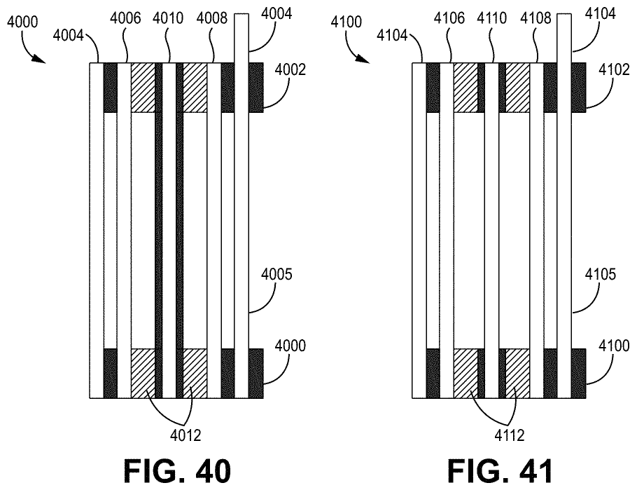

FIGS. 35A-C are a front view, an enlarged view, and a cross-sectional view of another example copper flex layer for use in the filtering portion described with respect to FIGS. 33 and 34. In this example, the copper-flex layer is similar to copper-flex layer 3308, except the copper-flex layer shown in FIGS. 35A-C has a plurality of grooves 3502 defined in the copper surfaces 3504 of the copper-flex layer. The copper layers 3504 are on both sides of a layer of polymer 3506 as described above with respect to copper flex layer 3308. The surfaces can have copper (other antiviral material as described herein) across the top, sides, and bottom of grooves. That is, all exposed surfaces in the area having the grooves can be composed of copper. The grooves increase the surface area of the copper and provide roughness which can aid causing the pathogens in the air to come into contact with the surface. Although only a single groove is shown in FIG. 35B it should be understood that multiple grooves can be included. In an example, parallel grooves are included (e.g., on substantially all) of both exposed copper surfaces of the copper flex layer. In an example, the grooves are less than 100 microns deep, such as 10 microns deep in an 18 micron thick copper layer. The grooves can be less than 500 microns wide, or less than 250 microns wide or less than 100 microns wide. In an example, the grooves are 75 microns wide. In an example, adjacent grooves are less than 500 microns apart, less than 250 microns apart, less than 100 microns apart. In an example, the grooves are less than 75 microns apart. Other dimensions are also possible for the grooves.

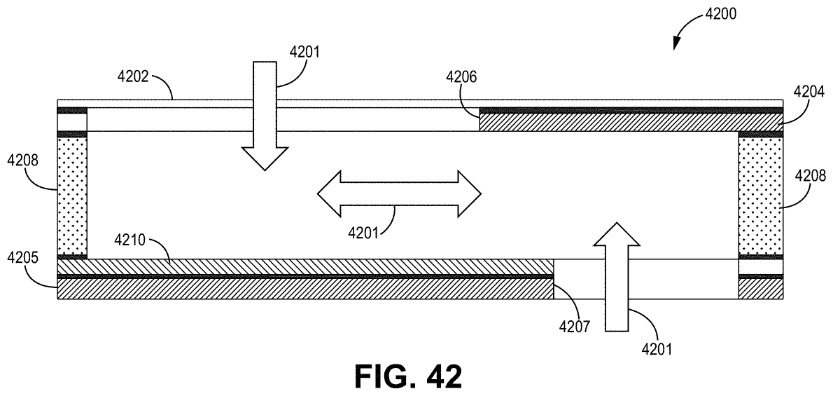

FIG. 36 is another example of a base frame 3602 of a respirator having a fit tester port 3604. A relationship has been established with TSI who is the world supplier of test instruments that are used to validate the N95 particle filtration efficacy of the filter materials, as well as an instrument called a fit tester. The fit tester is used to validate the fit and seal of the respirator while in place on the user's face. This is a cumbersome process and is rarely used each and every time a respirator to put on which defeats much of the effectiveness with air leakage around the edges. Accordingly, a fit test port can be included such that the user can fit the respirator and easily test the effectiveness of the fit and seal with a fit tester, such as the one manufactured by TSI.

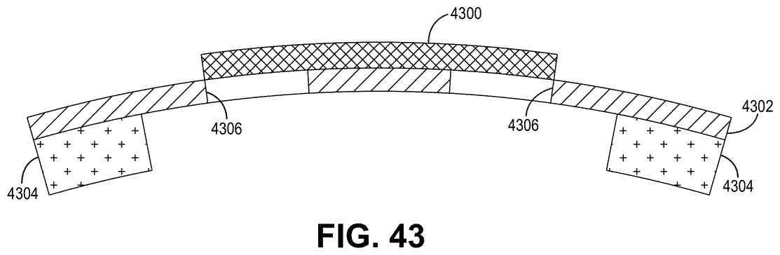

FIGS. 37A-D are perspective views of example detachable pivoting clips 3702, 3704 for detachably connecting head straps to the base frame 2602. FIG. 37A is a perspective view of the male clip 3704 connected to the female clip 3702. FIG. 37B is a perspective view of the female clip 3702 and FIG. 37C is a perspective view of the male clip 3704. FIG. 37D is a side view of the male clip 3704 showing the connecting knob 3706. The female clip 3702 can be removably connected to one of the strap connectors 2608 on the base frame 2602. The male clip 3704 can be attached to a strap by weaving the strap through a strap pincher 3708 (or other strap connector). The knob 3706 of the male clip can be inserted into an aperture 3710 of the female clip 3702 to detachably connect them together. In an example the knob 3706 and/or aperture 3710 are generally rounded to enable the male slip 3704 to rotate with respect to the female clip 3702. This provides instant adjustment to the user's head and face and easy removal of straps for sanitation. This also enables the straps to be disconnected while wearing the respirator instead of pulling the strap over the head.

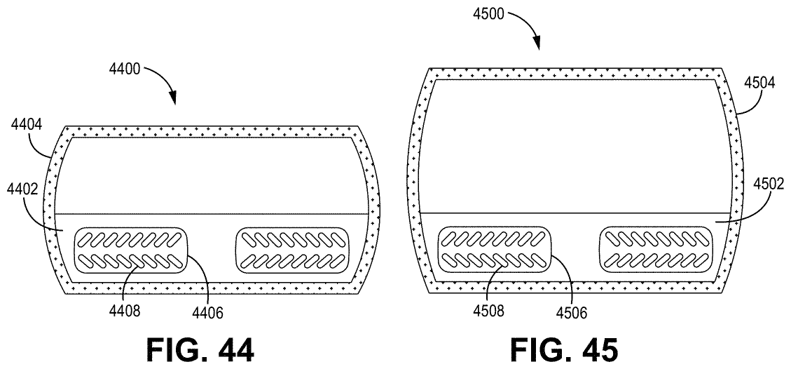

FIGS. 38A-D are illustrations of an example filter cartridge 3800 for use in any of the cartridge respirators described herein, including with base frame 2602. The cartridge 3800 includes a molded support member that defines one or more apertures 3804 for air flow therethrough. In this example, the support member defines an outer ring 3808 and an inner hub 3810. The one or more apertures 3804 are defined between the outer ring 3808 and the inner hub 3810.

A filter material, such as a N95 type filter material, is disposed across the apertures 3804 to filter air in both directions entering and leaving the respirator. In this example, the filter material has a generally circular shape and extends from the inner hub 3810 to the outer ring 3808 to cover all the apertures 3804. The filter material can be heat sealed to the support member. The interior of the support ring is shown with six (6) thin webs to maximize air flow area and provide some theoretical compliance around the ring if loaded against a gasket-mask face seal.

The cartridge also defines a cartridge securing member that is configured to removably secure the cartridge to the base frame 2602. In an example, the cartridge securing member is a physical structure that removably interlocks with a corresponding structure on the base frame 2602 of the facepiece. In other examples, other cartridge securing members can be used, such as an adhesive, magnet, or Velcro type feature. In this example, the cartridge securing member is a ‘T’ stub feature 3816 extending therefrom that mates with a corresponding female slot 2606 on the base frame 2602. The T stub 3816 and corresponding slot 2602 are configured such that the cartridge can be removably secured to the base frame 2602 by inserting a T stub 3816 into the slot 2602 and rotating the cartridge relative to the base frame 2602 to lock the taps of the T stub 3816 into the slot 2606. A gasket can be located around the perimeter of the outer ring 3808 to seal against the base frame 2602 surface during the twist action that is intended to pull the ring towards the surface of the base frame 2602. Advantageously, a single base frame 2602 can be configured to have multiple different types of filter cartridges attached thereto, such that the particular cartridge for an application can be selected and installed. The twist to install filter cartridges discussed herein also enable the filter to be easily attached to the base frame 2602 by hand.

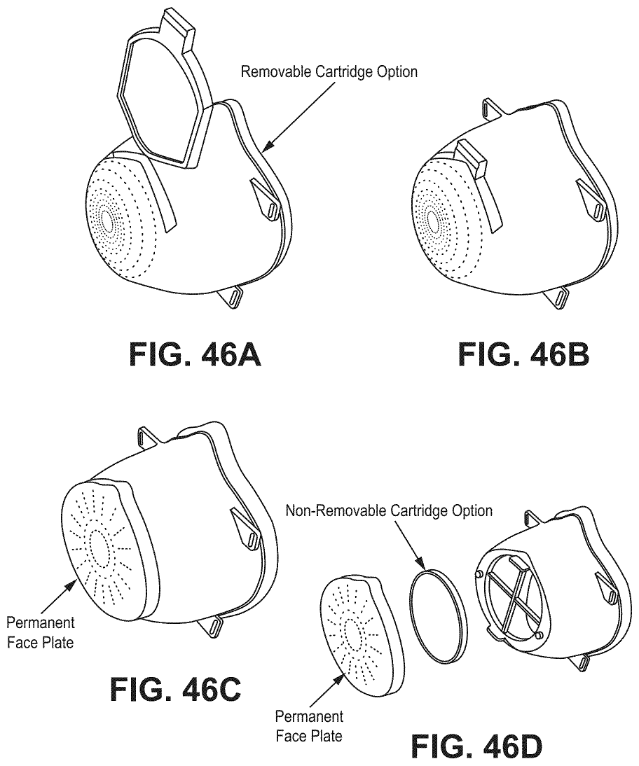

FIG. 39 is an exploded view of another example cartridge 3900 for use in any of the cartridge respirators described herein, including with base frame 2602. The cartridge 3900 includes a molded outer support member 3902 that defines one or more apertures 3904 for air flow therethrough. The outer support member 3902 defines an outer ring and an inner hub. The one or more apertures 3904 are defined between the outer ring and the inner hub.

The cartridge 3900 also includes middle retainer 3908 and back retainer 3910. The support member 3902 and retainers 3908, 3910 can be composed of a rigid material (e.g., polymer) and provide support for a plurality of filtering and anti-pathogenic layers. The filtering layers can include an outer layer 3912 and inner layer 3914 of filter material, such as a N95 type filter material, that is disposed across the apertures 3904 to filter air in both directions entering and leaving the respirator. In an example, the filter layers 3912, 3914 can be composed of 25 gram per square meter melt blown material. The filter layers 3912, 3914 can have a generally circular shape and extend to cover all the apertures 3904. The filter material can be heat sealed to the adjacent member 3902 and retainers 3912, 3914. The interior of the support ring is shown with six (6) thin webs to maximize air flow area and provide some theoretical compliance around the ring if loaded against a gasket-mask face seal. The inner retainer 3908 provides separation between the filtering layers 3912, 3914 which can be a significant benefit in effectiveness vs material in contact with each other. In addition, this allows for multiple layers of less restrictive filter materials to be used as an alternative to a single layer of a more restrictive material. For example, two (2) layers of 25 gram per square meter melt blown material that is separated can provide a more breathable and effective filtration than 1 layer of 50 GSM material.

The cartridge 3900 can also include one or more anti-pathogen layers 3918 that have pathogen destroying metallization thereon. The anti-pathogen layer 3918 can be constructed of bare elemental metal such as copper or can be a sheet of polymer having anti-pathogen (e.g., metal) layers thereon and flaps defined therein as discussed herein to provide surfaces for anti-pathogen materials to contact as the air flows through the cartridge 3900. Use of such an anti-pathogen layer significantly increases the probability of a pathogen encountering the surface in compared to methods of filament, fibers, threads or particles that are spaced apart in relatively large distance. Example anti-pathogen layers are described as baffles in PCT Patent Application No. PCT/US2022/014018, filed on Jan. 27, 2022, and entitled “FACEPIECE INCLUDING AIRFLOW BAFFLE WITH AN ANTIPATHOGEN SURFACE”, which hereby incorporated herein by reference.

The cartridge also defines a cartridge securing member that is configured to removably secure the cartridge to the base frame 2602. In an example, the cartridge securing member is a physical structure that removably interlocks with a corresponding structure on the base frame 2602 of the facepiece. In other examples, other cartridge securing members can be used, such as an adhesive, magnet, or Velcro type feature. In this example, the cartridge securing member is a ‘T’ stub feature 3916 extending from the outer support member 3902 that mates with a corresponding female slot 2606 on the base frame 2602. The T stub 3916 and corresponding slot 2602 are configured such that the cartridge can be removably secured to the base frame 2602 by inserting a T stub 3916 into the slot 2602 and rotating the cartridge relative to the base frame 2602 to lock the taps of the T stub 3916 into the slot 2606. The T stub 3916 can extend through each of the filter layers 3912, 3914, and pathogen layer 3918. A gasket can be located around the perimeter of the back retainer 3910 to seal against the base frame 2602 surface during the twist action that is intended to pull the ring towards the surface of the base frame 2602. Advantageously, a single base frame 2602 can be configured to have multiple different types of filter cartridges attached thereto, such that the particular cartridge for an application can be selected and installed. The twist to install filter cartridges discussed herein also enable the filter to be easily attached to the base frame 2602 by hand.

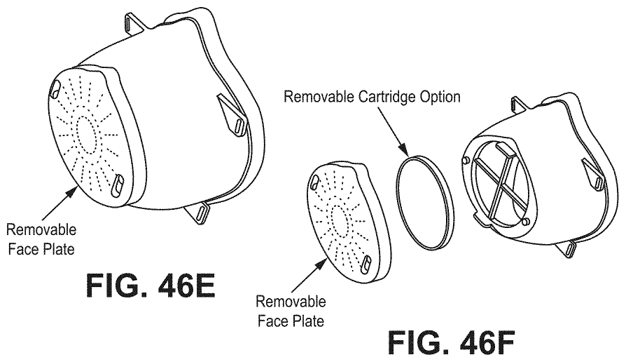

FIGS. 40 and 41 are cross-sectional views of two example filters 4000, 4100 that are adhered over one or more apertures in a facepiece. The filters 4000, 4100 can have a tape-like flexible structure with a peelable adhesive 4002, 4102 on portions of one side thereof. The adhesive 4002, 4102 can enable the filters 4000, 4100 to be detachably adhered to a surface of a facepiece. Each filter 4000, 4100 can have a plurality of layers. The filter 4000 of FIG. 40 includes two barrier layers 4004, 4005, two filter layers 4006, 4008, and an anti-pathogen layer 4010 with spacers 4012 in between. The filter 4100 of FIG. 41 includes two barrier layers 4104, 4105, three filter layers 4106, 4108, 4110 with spacers 4112 in between. A tab 4014, 4114 can be included to aid in peeling the filter 4000, 4100 off of the facepiece. Example filter structures to which a peelable adhesive can be applied are described in PCT Patent Application No. PCT/US2022/014018, filed on Jan. 27, 2022, and entitled “FACEPIECE INCLUDING AIRFLOW BAFFLE WITH AN ANTIPATHOGEN SURFACE”.

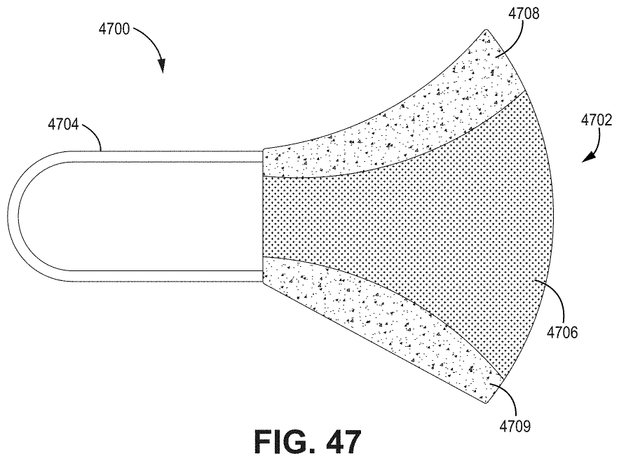

FIG. 42 is a cross-sectional view of another tapeable filter 4200. Filter 4200 defines an air flow path 4201 such that air travel is forced to encounter metallized baffles within the filter 4200. This approach allows for large airflow pathways that redirect airflow and force moisture bearing droplets to hit the metal surfaces and deposit the pathogens onto the surface while creating less resistance to airflow. Filter 4200 includes outer PET extruded plastic carriers 4204, 4205 that define respective apertures 4206, 4207 into and out of filter 4200. A layer of GSM material 4202 can be disposed over the aperture 4206. Foam adhesive 4208 can be disposed between the PET carriers 4204, 4205 to provide space for the airflow path 4201 therebetween. A layer of metal 4210 (e.g., copper or any of the other metal layers described herein) can be disposed on one or more interior surfaces of the airflow path.

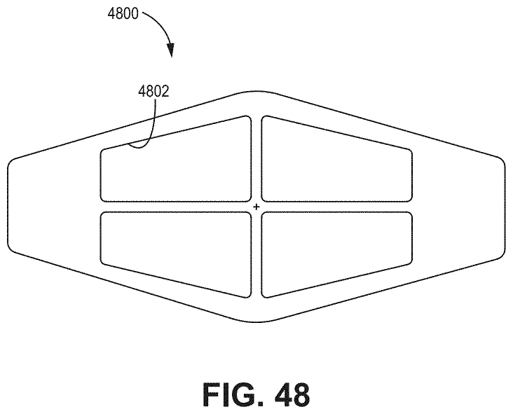

FIG. 43 is a cross-sectional view of yet another tapeable filter 4300. Filter 4300 can be applied to respirators that resemble a face shield commonly used in medical environments to protect the user and the respirator or surgical mask they are wearing from splashes. The face shield can be constructed of a sheet of polymer 4302 such as polycarbonate, polyester, acrylic, etc. that has conformal foam 4304 mounted to the perimeter to seal against the face and regions cut for airflow and filter tape placement. The face shield can define one or more airflow regions 4306, wherein the tapeable filter 4300 covers the airflow regions 4306. The face shield respirator can be constructed to seal at the nose and cheek areas or elongated to seal at the forehead. Airflow regions that accept the tapeable filter 4300 can be any size and shape and located strategically on the surface to maximize airflow, filtration, or pathogen destruction and reduce the potential for fogging.

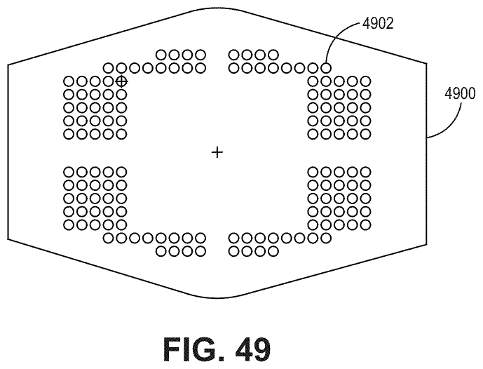

FIGS. 44 and 45 are front view of other example face shields 4400, 4500 having a tapeable filter 4402, 4502 thereon. Each face shield 4400, 4500 can have compliant foam 4404, 4504 around the edges thereof and a tapeable filter 4402, 4502 extending over one or more apertures 4406, 4506 therein. The tapeable filters 4402, 4502 can define a plurality of airflow paths 4408, 4508 as described with respect to FIG. 42.

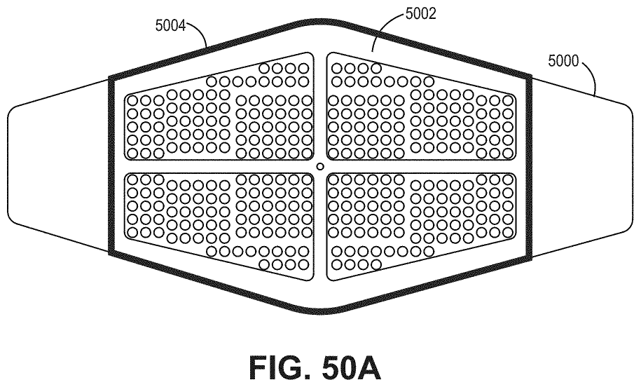

FIGS. 46A-F are other example respirators having slide-in cartridges that can be secured thereto. The cartridges can include any of the filtering and antiviral aspects described herein.

Most consumer product enhancements claim anti-microbial properties with coatings, nano-particles, or metallic filaments or threads incorporated into the fabric. These methods do have some potential benefits in destroying microbes and bacteria with limited effect on viruses and more lethal pathogens. There are no respirator products that add anti-pathogen function with commercially available products focused on filtration standards.

The respirator can provide dramatic improvements to the user experience of wearing a respirator while adding a unique technology approach to pathogen destruction beyond simple filtration. The respirator incorporates a generally planar surface or surfaces on the cartridge that bear anti-pathogen metallization that is strategically located within the airstream of inhale or exhale or both. This planar member can be described as an electrical circuit like member, as it can be a passive structure that simply bears metallization or coatings that disable or destroy a virus or pathogen that encounters the surface, or it can be an active circuit member that provides a platform for electrification of circuits or powering devices.

The anti-pathogen circuit or anti-pathogen surface placed within the airflow path in some fashion has significant advantages over the methods used to incorporate particles, threads, or coatings within a cloth consumer face mask. Those methods have a relatively small density of anti-microbial or antiviral material relative to the actual material content of the mask and corresponding airflow volume. In other words, the vast majority of the airflow and airborne pathogens pass through the untreated areas of the fabric. The use of a surface bearing anti-pathogen properties significantly increases the probability of any airborne pathogen encountering the surface and remaining in a disabled state no longer able to infect or replicate. Since the airborne pathogen are essentially carried by moisture droplets large and small, the surface can be enhanced to promote pathogen capture and prolong the duration of direct contact with the anti-pathogen measures.

In an example, the antiviral material described in any of the examples herein can include copper, silver, zinc or a combination thereof. In an example, the antiviral material is zinc plus copper, wherein there is a base copper layer with a nickel barrier and zinc over the nickel. Portions of the copper are exposed alongside exposed zinc creating an oxidation reaction between the two substances in the presence of moisture (e.g., water droplets). The nickel is used to plate the zinc on the copper in a way that reduces attach of the copper by the zinc. In another example, a base zinc layer is used with copper added to the zinc and having exposed copper next to exposed zinc.

In some examples, a saline can be applied over the exposed copper and zinc and then dried so that dried salts reside on the exposed copper and zinc surfaces. While the surfaces remain dry, the oxidation reaction is paused, and the salt remains dried. As the surface is exposed to moisture (e.g., a user's breath) the salt dissolves and jump starts the metal free ion exchange during oxidation creating a mild voltage self-generating battery effect. This can be highly effective at disabling viruses. The saline adds sodium and chloride ions that drive the oxidation corrosion which is the basic chemical reaction that destroys the virus's ability to replicate.

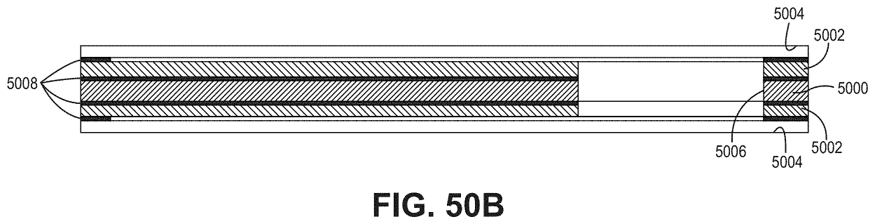

FIG. 47 is a side view of another example mask 4700. Mask 4700 is composed primarily of fabric, with a unique structure that creates an air chamber near the face for significantly improved breathability. The mask 4700 also restricts and directs airflow through targeted areas and can optionally contain an antipathogen planar structure suspended within the airflow path. The antipathogen structure is a metal bearing circuit type member that is processed to enhance the pathogen destruction properties and mounted within the fabric shell to present an antipathogen environment contained within the filtration structure such that when the pathogens within the airflow encounter the antipathogen structure the pathogens are disabled. The filtration mechanism consists of a polymer stiffener that resides within the fabric in such a way that airflow is restricted and directed through filtration regions containing N95 type filter materials while providing a much better seal to the face to provide protection beyond conventional fabric and surgical masks. The polymer stiffener can be sewn directly into the fabric to add the air chamber feature which suspends the fabric material away from the user's face while enhancing the airflow characteristics.

Traditional facial masks used for exposure situations range from basic cloth patches that cover the mouth and nose, surgical masks, to more elaborate respirator formed structures that have fibrous structures that create a filter effect for air that is breathed in by the user as well as exhaled by the user. In some cases, the filter effect is directed at protecting the user from inhaled pathogens or contaminants, and in some cases the intent is to prevent the user from exhaling pathogens or infectious particles. In general, most commercially available respirators are intended for a one time use and discarded, and are difficult or expensive to sterilize and return to as new condition. In addition, most if not all commercially available masks and respirators are designed and used to reduce exposure to pathogens and do not attack the potential pathogens themselves. The present invention is aimed at providing a basic pathogen exposure protection in a mask or respirator that can be easily sterilized and reused, as well as optionally extend to destruction of pathogens and further extension to active embedded electronics that can provide extensive pathogen abatement and provide extensive protection to the user and surrounding persons. The N95 respirator is the conventional standard in medical and healthcare type settings, with challenges to the user with regard to fit, seal to the face and comfort as well as sanitation. Cloth masks worn by the general public provide better protection than no mask at all, but the cloth fibers do little to filter pathogens or protect the user or those around the user from infection. Several companies have introduced masks that contain anti-microbial materials and some products include copper filaments or zinc-oxide particles or silver-zinc particles to increase the potential of virus disablement. These products are a step beyond plain cloth masks and are not intended for medical or healthcare type situations, while N95 respirators remain the baseline for healthcare. In many cases, users wear a face shield or 2^{nd} surgical mask over the N95 respirator to provide added protection as well as assist with keeping the underlying respirator from external contamination as it is common to wear the respirator beyond the 1 time use recommendation. The present invention is aimed at providing a much better version of a respirator or face mask that can be reused, sanitized, returned to new condition, and add anti-pathogen properties to extend protection beyond simple filtration. A feature of the construction is the addition of a polymer structure internal to the mask such that an air chamber is created in front of the mouth and nose of the user to dramatically increase airflow characteristics while holding the material away from face contact.

Mask 4700 includes a main body 4702 and one or more straps 4704 extending therefrom. The strap(s) 4704 can be configured to extend around the ears or the head of the user to hold the mask 4700 onto the face. The straps 4704 can be composed of any suitable material, including elastic and/or non-elastic materials. The main body 4702 can be composed of a plurality of fabrics and have a generally non-rigid structure. That is, the fabrics of the main body 4702 will generally conform to the geometry of the user's face or as otherwise manipulated. The main body 4702 can include one or more areas 4706 of high airflow material and one or more areas 4708, 4709 of low airflow material. In the example shown in FIG. 47, the high airflow areas 4706 are positioned vertically in the middle of the main body 4702 with strips 4708, 4709 of no flow material extending horizontally across the top and bottom of the main body 4702.

The high 4706 and no-flow 4708, 4709 materials provide good protection to the user by restricting airflow from undesired areas while driving airflow through areas with filtration areas that use filtering materials such as melt blown N95 type filter fabric. The upper 4708 and lower 4709 no-flow strips block airflow and can optionally be composed of an elastic material like neoprene to aid with fit and seal to the face while preventing airflow through these areas 4708, 4709.

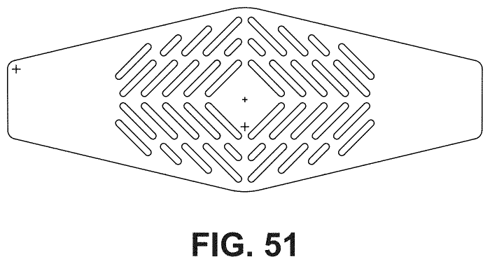

FIG. 48 is a front view of an example stiffener 4800 that can be included in the main body 4702 to provide a geometric shape that holds the main body 4702 away from a user's face. The main body 4702 can define a pocket that corresponds to the profile of the stiffener 4800, such that the stiffener 4800 can be placed into the pocket and be held in the main body 4702. The pocket and stiffener 4800 can be configured such that the stiffener 4800 extends horizontally across the face of the user in front of the user's nose and mouth. When in the pocket, the stiffener 4800 can provide a general geometry that is vertically flat and horizontally arced away from a user's face. The arc of the stiffener 4800 when in the pocket can have a smaller radius than the generally horizontal arc of a user's face in the mouth area and the length of the arc of the stiffener 4800 in the pocket can be longer than the arc of the user's face to hold the main body 4702 away from the user's mouth. In an example, the stiffener 4800 can have a naturally planar geometry, and is configured of a flexible material, such as a polymer, that forms an arc when placed into the pocket. The pocket can be configured to cause the arc to be formed by having edges that are slightly closer together if one were to draw a horizontal straight line therebetween, than the horizontal length of the stiffener 4800. This causes the stiffener 4800 to form an arc shape when forced into the pocket with the two edges.

The stiffener 4800 can be manufactured using any suitable method such as punching laser cutting, molding, etc. The stiffener 4800 can be installed into the main body 4702 as a removable and replaceable component or the stiffener 4800 can be sewn or otherwise permanently fixed to the main body 4702. The stiffener 4800 is configured to be flexible enough to allow for bending to create the arc described above, yet stiff enough to spread out the geometry of main body 4702 by restraining edges or other features in such a way that forces the stiffener 4800 to spring and spread the main body 4702 to the desired location and geometry.

In an example, the stiffener 4800 is disposed primarily or entirely within the high airflow area. That is, the stiffener 4800 is covered with high airflow material. Accordingly, the stiffener 4800 defines one or more apertures 4802 to allow airflow therethrough, such that air can enter and exit the internal chamber created by the mask via the apertures(s) 4802.

In an example, additional filtration materials can be mounted to one or both sides of the stiffener 4800 and disposed over the aperture(s) 4802. The filtration materials can be mounted to the stiffener 4800 in any suitable manner including via adhesive, thermal or ultrasonic welding. Any suitable filtration material can be used including melt blown, non-woven or spun bond materials used in N95 respirator or surgical masks as well as a wide variety of materials.

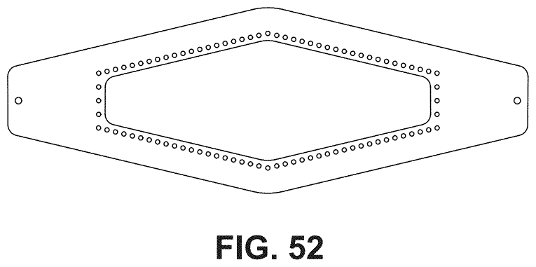

FIG. 49 is an example of an antipathogen panel 4900 that can be mounted to the stiffener 4800 and disposed over the aperture(s) 4802. The antipathogen panel 4900 can have antipathogenic surfaces thereon configured to disable pathogens in the airflow that is brought into contact therewith. The antipathogen panel 4900 can be composed of metal or a substrate, such as LCP, having metal on one or more surfaces thereof. Any of the example antipathogen metals or a combination thereof can be used, including copper, silver, zinc, zinc oxide, and nickel. The antipathogen panel 4900 can be disposed to disrupt the straight through airflow and drive a large percentage of the airflow against a metalized surface. Pathogens encountering the antipathogen panel 4900 will disperse from moisture droplets and enter a free ion exchange chemical reaction environment that disables the virus and its' ability to replicate or infect the user. The metal surface(s) of the antipathogen panel 4900 can also be enhanced with treatments such as saline to increase the chemical reaction potency or other coatings that are destructive to pathogens but not harmful to the user. Any example airflow features 4902 can be included in the antipathogen panel 4900 to allow airflow therethrough, such as aperture(s), flaps, or a combination thereof.

In an example, the antipathogen panel 4900 can define a plurality of airflow torture paths therethrough in accordance with any of the examples described herein. In an example, multiple distinct antipathogen panels 4900 having varied airflow feature patterns can be positioned in series to provide an airflow torture path through the set of panels.

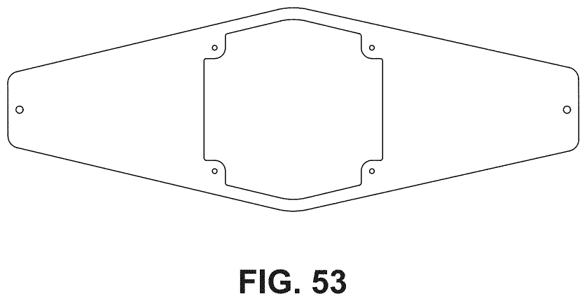

FIG. 50A is a front view of an example stiffener 5000 having one or more antipathogen panels 5002 along with one or more layers of filtering material 5004 (only the edge is shown) mounted to the stiffener 5000 to provide both filtering and antipathogenic properties. FIG. 50B is a cross-sectional view of a portion of the example stiffener 5000 showing an antipathogen panel 5002 on each side and a layer of filtering material 5004 on the antipathogen panels 5002 and extending over the aperture(s) 5006 defined in the stiffener 5000 and antipathogen panels 5002.

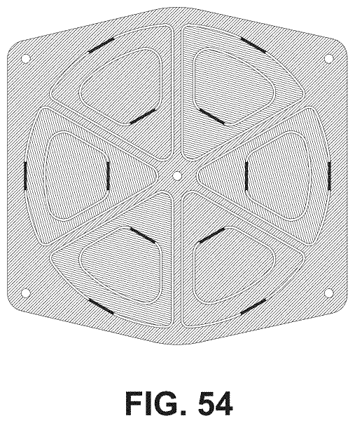

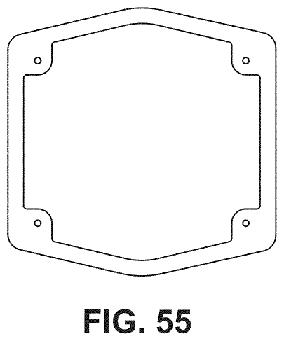

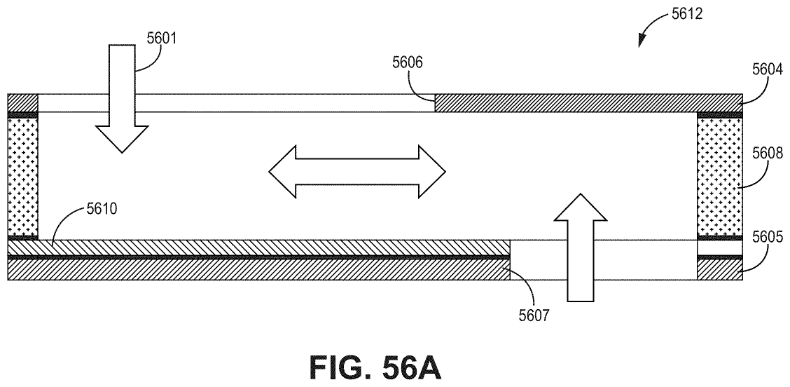

FIGS. 51-53 are a front views of other example stiffeners. FIG. 54 is a front view of another example antipathogen panel defining flaps therein and having microfluidic channels defined in the metal antipathogen surfaces. FIG. 55 is a front view of an example spacer frame that can be disposed on top of the antipathogen panel of FIG. 54. A layer of filtering material can be disposed on top of the spacer frame, wherein the spacer frame acts to provide space between the antipathogen panel on and the layer of filtering material. Such a stack of antipathogen panel, spacer, and filtering material can be disposed on one or both sides of the stiffener. The antipathogen panel can have include any of the airflow defining features described herein and/or any of the features described as baffles in PCT Patent Application No. PCT/US2022/014018, filed on Jan. 27, 2022, and entitled “FACEPIECE INCLUDING AIRFLOW BAFFLE WITH AN ANTIPATHOGEN SURFACE”, which hereby incorporated herein by reference.

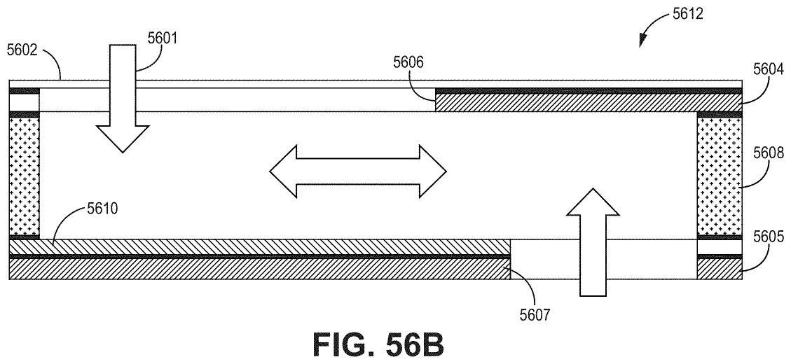

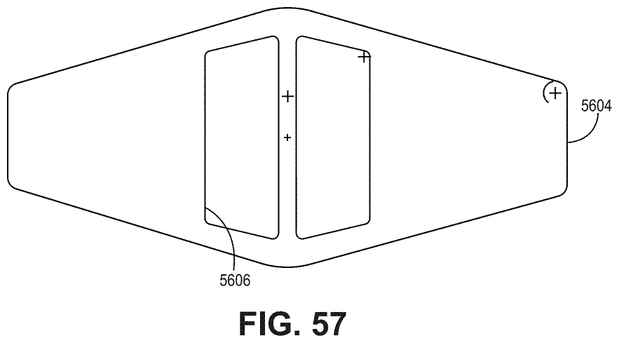

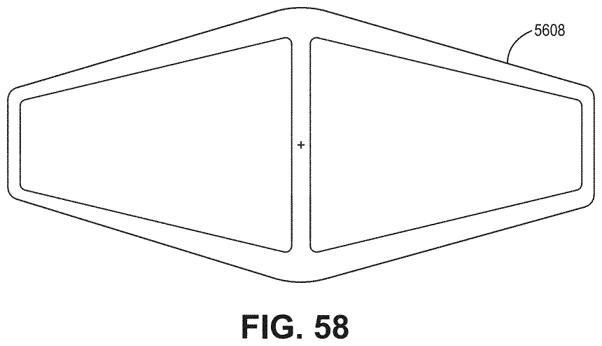

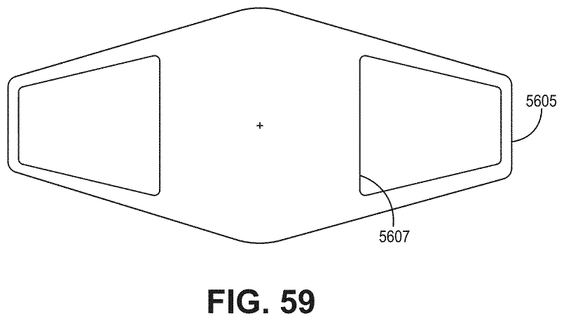

FIGS. 56A and 56B are cross-sectional views of portions of other example stiffeners 5600, 5612. FIG. 56A shows stiffener 5600 defines an air flow path 5601 such that air travel is forced to encounter metallized baffles within the stiffener 5600. This approach allows for large airflow pathways that redirect airflow and force moisture bearing droplets to hit the metal surfaces and deposit the pathogens onto the surface while creating less resistance to airflow. Stiffener 5600 includes outer PET extruded plastic carriers 5604, 5605 that define respective apertures 5606, 5607 into and out of stiffener 5600. Foam adhesive 5608 can be disposed between the PET carriers 5604, 5605 to provide space for the airflow path 5601 therebetween. A layer of metal 5610 (e.g., copper or any of the other metal layers described herein) can be disposed on one or more interior surfaces of the airflow path. FIG. 56B shows stiffener 5612, which is identical to stiffener 5600, except stiffener 56A has a layer of GSM material 5602 disposed over the aperture 5606. FIG. 57 is a front view of an example inner carrier 5604. FIG. 58 is a front view of an example foam adhesive layer 5608. FIG. 59 is a front view of an example outer carrier 5605.

In example, a tape like filter, such as those described with respect to FIGS. 40-45 herein can be applied to a stiffener.

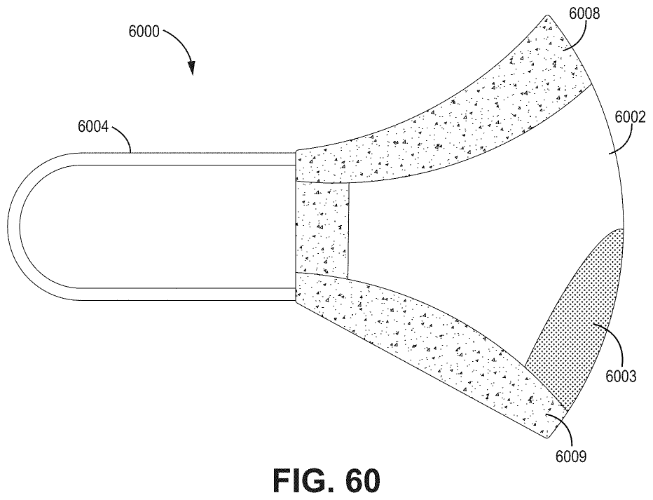

FIG. 60 is a side view of an example mask 6000 that includes a clear panel 6002 similar to face shields commonly used in medical environments to protect the user and the respirator or surgical mask they are wearing from splashes. The clear panel 6002 can be constructed of a sheet of polymer, such as a polycarbonate, polyester, acrylic, etc. The clear panel 6002 can be integrated with the fabric of the main body and region(s) defined in the clear panel 6002 for placement of a tape like filter 6003 in front of the user's nose/mouth. The clear construction allows the user's face to be visible. The mask 6000 can include straps 6004 and no flow regions 6008, 6009 (e.g., composed of neoprene) on the top and bottom portions thereof as discussed above. The clear panel 6002 can be bonded or stitched to the neoprene and springs to shape based upon the restricts and design contour to hold the mask 6000 away from the user's mouth. Airflow is directed to the filter 6003 which can be center or off-center, or around the perimeter of the clear polymer panel 6002. The clear panel 6002 can be coated to reduce fogging due to moisture in user's breath. To aid in fit to face, compliant foam, which can be molded by hand and maintain its shape, can be added to the neoprene panels in areas that tough the face (e.g., along the top and bottom no-flow strips).

What is claimed is:

1. A facepiece comprising:
a main body having a geometry configured to fit on a human face and cover the human's mouth and nose, the main body defining an interior cavity between the main body and the human's face; and
one or more straps extending from the main body and configured to extend around the ears or head of a human such that the main body is held on the human's face,
wherein the main body includes a filter defining a plurality of airflow torture paths extending therethrough between the interior cavity and an external environment, wherein the filter includes three or more layers of airflow path defining features, each airflow path defining feature providing a passageway for air to pass therethrough, wherein the path defining features in adjacent layers are fluidly coupled but offset to form the airflow torture paths.

2. The facepiece of claim 1, wherein each layer of the three or more layers of airflow path defining features is less than 50 microns thick.

3. The facepiece of claim 1, wherein portions of the airflow torture paths are smaller than 5 microns across.

4. The facepiece of claim 1, wherein portions of the airflow torture paths are smaller than 1 micron across.

5. The facepiece of claim 1, wherein the facepiece meets the N95 standard in force by the United States National Institute of Occupation Safety and Health (NIOSH) on Jan. 1, 2021.

6. The facepiece of claim 1, wherein the three or more layers includes ten or more layers of airflow path defining features disposed such that airflow path defining features in adjacent layers are fluidly coupled but offset to form the airflow torture paths.

7. The facepiece of claim 1, wherein the main body is composed primarily of a monolithic structure of LCP, wherein the filter is a portion of the monolithic structure defining the plurality of tortious passageways.

8. The facepiece of claim 7, wherein more than half of the surface area of the monolithic structure is impervious to air, such that no airflow paths are defined therethrough.

9. The facepiece of claim 7, wherein the filter encompasses more than half of the surface area of the monolithic structure.

10. The facepiece of claim 1, wherein the filter is removably attached to a remainder of the main body.

11. The facepiece of claim 1, wherein the airflow path defining features include apertures that are less than 25 microns across.

12. The facepiece of claim 11, wherein apertures within each layer of the three or more layers are less than 100 microns apart from other apertures in their own layer.

13. The facepiece of claim 11, wherein adjacent layers the three or more layers are bonded directly together.

14. The facepiece of claim 11, wherein the filter includes a bond layer bonded between two or more adjacent layers of the three or more layers.

15. The facepiece of claim 11, wherein the three or more layers of airflow path defining features are unbonded in an area including the airflow path defining features and are secured together in an area outside of the airflow defining features.

16. The facepiece of claim 11, wherein the filter includes one or more of copper and silver plating on an interior surface of the apertures.

17. The facepiece of claim 16, wherein the filter includes circuit traces electrically coupling the one or more copper and silver plating to a power source.

18. The facepiece of claim 16, wherein the filter includes circuit traces electrically coupling plating in adjacent apertures together.

19. The facepiece of claim 1, wherein the airflow path defining features include flaps that are configured to flex in response to pressure change during a human's breathing.

20. The facepiece of claim 19, wherein adjacent layers of flaps are spaced apart to provide room for the flaps to deflect.

21. The facepiece of claim 19, wherein the flaps have a freely hanging end.

22. The facepiece of claim 19, wherein the flaps are fixed at two locations opposite one another and the flaps are configured to bend in between and/or rotate about the two fixed locations.

23. The facepiece of claim 19, wherein the filter includes copper or silver plating on one or both surfaces of the flaps.

24. The facepiece of claim 1, wherein one or more layers of the three or more layers of airflow path defining features defines an anti-microbial surface aligned with the airflow path defining features of an adjacent layer such that airflow through the airflow path defining features of the adjacent layer is incident on the anti-microbial surface, wherein the anti-microbial surface has one of a copper or silver plating thereon.
